(19) United States
(12) Patent Application Publication
(10) Pub. No.: US 2007/0243159 A1
(43) Pub. Date: Oct. 18, 2007

(54) Therapeutic Compositions and Vaccines By Glycosyl-Phosphatidylinositol (Gpi)-Anchored Cytokines and Immunostimulatory Molecules

(76) Inventor: Periasamy Selvaraj, Lilburn, GA (US)

Correspondence Address: Zhaoyang Li;Squire Sanders & Dempsey, Sutie 300, One Maritime Plaza, San Francisco, CA, 94111, US

(21) Appl. No.: 10/554,609

(22) PCT Filed: Apr. 30, 2004
(86) PCT No.: PCT/US04/13931

§ 371 Date: Feb. 26, 2007

Related U.S. Application Data
(60) Provisional application No. 60/466,827, filed on Apr. 30, 2003.

(51) Int. Cl. A61K 39/00; A61K 39/12

(52) U.S. Cl. 424/85.2; 424/204.1; 424/277.1; 424/85.1

(57) ABSTRACT

A therapeutic composition or a vaccine comprising tumor membrane-anchored cytokines or other immunostimulatory or costimulatory molecules are provided. The therapeutic composition or a tumor vaccine can be used for treating a tumor or other disease such as autoimmune disorder, viral diseases, bacterial diseases, parasitic diseases, and transplant rejection.

### BACKGROUND OF THE INVENTION

### Field of the Invention

This invention generally relates to a tumor vaccine formed from one or more membrane-anchored cytokines or immunostimulatory molecules. Description of the Background Cytokines play a crucial role in induction of antitumor immune response (Pardoll, D. M., 13: 399-415 (1995); Trinchieri, G., Eur Cytokine Netw., 8: 305-7 (1997); and Mach, N. and Dranoff, G., Curr Opin Immunol., 12: 571-575 (2000)). Preclinical studies have demonstrated that administration of cytokines such as IL-2, IL-4, IL-6, or IL-12, induce stimulation of antitumor immune responses (see, for example, Rosenberg, S. A., et al., J Exp Med., 161: 1169-88 (1985)). For example, studies in murine tumor models have demonstrated, for instance, that antitumor immune responses can be stimulated post administration of the cytokines IL-2 (Rosenberg, S. A., 3 Natl Cancer Inst 75:595-603 (1985)), IL-4 (24), IL-6 (Brunda, M J, et al., J Exp Med 178:1223-1230 (1993); Nastala, C. L., et al., J Immunol 153:1697-1706 (1994)), or granulocyte-macrophage colony stimulating factor (GM-CSF) (Dranoff, G., et al., Proc Natl Acad Sci USA 90:3539-3543 (1993); Hung, K, et al., J Exp Med 188:2357-2368 (1998)). IL-2, one of the chemical mediators of the immune response, has been shown to have antitumor capabilities through its activation of helper (Mosmann, T. R., et al., J Immunol 136:2348-2357 (1986)) and cytotoxic T cells (McAdam, A. J., et al., Cell Immunol 165:183-192 (1995)), natural killer (NK) cells (Trinchieri. G., Biology of Natural Killer Cells. Adv Immunol 147:187-303 (1989)), lymphokine activated killer (LAK) cells (Rosenberg, S. A., J Natl Cancer Inst 75:595-603 (1985)), and macrophages (Baccarini, M., et al., J Immunol 142:118-125 (1989)). IL-12 attracts T cells, APCs, NK cells, and inflammatory cells to the site of secretion or vaccination and can also activate and enhance the maturation of antigen-specific cytotoxic T cells (CTLs) (Trinchieri, G and Scott P., Curr Top Microbiol Immunol 238:57-78 (1999)).

Systemic administration of cytokines to humans, particularly IL-2, initially appeared to have promising results (Rosenber, S. A., et al., Ann Intern Med 108:853 (1988); Lotze, M. T., et al., J Am Med Assoc 526:3117-3124 (1986); and Rosenberg, S. A., et al., N. Eng J Med 319:1676 (1988)); however, systemic administration of the IL-2 to humans is problematic, not only because of rapid degradation (Lotze, M. T., et al., J Immunol 135:2865-2875 (1985)), but also because of severe toxic side effects due to paracrine activity (Siegel, J. P. and Puri, R. K., J Clin Oncol 9:694-704 (1991)). Leonard and co-workers (Leonard, J. P., et al., Blood 90:2541-2548 (1997)) found that systemic delivery of IL-12 is also highly toxic to patients, depending on the cytokine administration schedule. To circumvent the negative side effects associated with systemic cytokine administration, researchers developed an ex vivo method of stimulating T cells (Rosenberg, f1985, supra; Rosenberg, S. A., et al., N. Eng J Med 319:1676 (1988); Mule, J, J., et al., Science 225:1487-1489 (1984); Yang, J. C. and Rosenberg S A. Current approaches to the adoptive immunotherapy of cancer. Adv Exp Med Biol 233:459-467 (1988)). High doses of cytokines such as IL-2 were used to produce LAK cells from T cells and NK cells, which were then administered to patients. This method was met with only minimal success, however, and it has recently been shown that neither the co-administration of systemic IL-12 nor GM-CSF improves the antitumor response (Rosenberg, S. A., et al., J Immunol 163:1690-1695 (1999)). Alternative methods have therefore been developed to use cytokines in antitumor immunotherapy.

An alternative approach to stimulating an antitumor immune response is through the direct use of APCs. Initial methods relied peptide-pulsed macrophages (Mukherji B and Macalister T J. J Exp Med 158:240 (1983)) and on cell fusion of APCs with tumor cells, resulting in antigen-specific immunogenic tumor cells (Guo Y, et al., Science 263:518-520 (1994)). Cell fusion with dendritic cells (DCs) in particular results in the strongest antitumor responses (Wang J, et al., J Immunol 161:5516-5524 (1998)). More recent attention has been given to immunization with active DCs armed with tumor antigens on their cell surface (Bhardwaj N., Trends Mol Med 7:388-394 (2001). Sources of antigens for DC loading include apoptotic cells, tumor cells, live cells, cell lysates, proteins, or antigens encoded by DNA or RNA (Fonteneau J F, et al., J Immunother 24:294-304 (2001)). It has also been demonstrated that heat shock proteins isolated from tumor cells act as potent adjutants in inducing an antitumor immune response by stimulating DC maturation and antigen presentation (Srivastava P K, et al., Immunity 8:657-665 (1998)). DCs are attractive candidates for tumor vaccine strategies because relatively few numbers of cells are able to potently stimulate T-cell activation (Dhodapkar M V and Bhardwaj N, J Clin Immunol 20:167-174 (2000)). Notably, DCs are able to prime both antigen-specific CD4+ T cells and CD8+ T cells (Fonteneau J F, et al., J Immunother 24:294-304 (2001)). Clinical studies have demonstrated some limited metastatic regression and increased T-cell immunity post DC-vaccination (Dhodapkar M V, et al., J Clin Invest 104:173-180 (1999); Banchereau J, et al., Cancer Res 61:6451-6468 (2001)).

Antitumor T-cell response is dependent not only upon interaction with the tumor peptide antigen and major histocompatibility complex (MHC) (Mueller D L, et al., Annu Rev Immunol 7:445-480 (1989)), but also upon a second costimulatory signal that comes from the adhesion-receptor ligand binding between the antigen-presenting cell (APC) and the T cell (Linsley P S, et al., J Exp Med 173:721-730 (1991)); Azuma M, et al., Exp Med 175:353-360 (1992); Gimmi C D, et al., Proc Natl Acad Sci USA 90:6586-6590 (1993)). Many tumor cells, while expressing MHC molecules, lack the immune costimulatory or adhesion molecules necessary for T-cell activation and subsequent initiation of a host immune response (see, for example, Townsend S E and Allison J P., Science 259:368-370 (1993)). Without the second, costimulatory signal, clonal anergy will result in the tumor-specific T-cell population (see, for example, Tan P, et al., J Exp Med 177:165-173 (1993)). To counteract the down regulation or lack of many secondary stimulation signals, researchers have shown that the expression of costimulatory and other immunostimulatory molecules by gene transfer induces antitumor immune responses (see, for example, Pulaski B A and Ostrand-Rosenberg S, Cancer Res 58:1486-1493 (1998)).

Direct vaccination of mice with tumor cells transfected with IL-2 genes has been shown to provide protective immunity against parental tumor challenge (Porgador A, et al., Int J Cancer 53:471-477 (1993)) and to cause tumor regression in mice (see, for example, Fearon E R, et al., Cell 60:397-413 (1990)). Tumors transfected with genes from other cytokines, such as GM-CSF and IL-12, can also induce antitumor immunity (see, for example, Dranoff G, et al. Proc Natl Acad Sci USA 90:3539-3543 (1993)). Many studies in the murine system have shown that the transfection of costimulatory molecules can induce an antitumor immune response (see, for example, Li Y, et al, J Immunol 153:421-428 (1994)). It has been shown that after the expression of B7.1 in the human renal carcinoma line RCC-1 via gene transfer, RCC-1 stimulates strong proliferation and differentiation signals to autologous T cells (Wang Y—C, et al., J Immunother 19:1-8 (1996)).

Gene transfer requires the use of viral vectors, however, which complicate the treatment strategy as antiviral host immune responses may prohibit multiple immunizations using the same vector (see, for example, Davis H L, et al. Hum Gene Ther 4:733-740 (1993)). Additionally, due to the difficulty in transfecting primary tumor lines, gene transfer requires the establishment of tumor cell lines, which is also a time consuming process. Phase III gene therapy studies of immunostimulatory molecule transfection in humans have shown that the limiting factors in the process were the isolation of cells from the primary tumor and the low frequency of gene uptake. Gene transfection is ultimately impractical for a clinical setting (Simons J W, et al., Hum Gene Ther 1997; 57:1537-1546 (1997)).

Other strategies, such as co-injecting tumors with fibroblasts secreting cytokines (Tahara, et al., Cancer Res., 54(1): 182-189 (1994), or biodegradable gelatin polymers encapsulated with cytokines with tumor cell preparations (Golumbek, P. T., et al., Cancer Res., 53: 5841-5844 (1993)) also only provided limited success.

Therefore, there is a need for new ways of preparing cancer vaccine that do not require gene transfer procedures.

The examples and embodiments of the present invention described below address above-described problems and needs.

### SUMMARY OF THE INVENTION

In some embodiments, described herein is a method of tumor treatment or tumor vaccination. The method generally comprises applying to a human being in need thereof a tumor therapeutic composition or tumor vaccine defined herein. The tumor therapeutic composition or tumor vaccine can be produced by protein transfer of glycosyl-phosphatidylinositol (GPI)-anchored immunostimulatory or costimulatory molecules (FIGS. 3 and 4). In one embodiment, the tumor therapeutic composition or tumor vaccine comprises a live tumor cell or tumor cell membranes that is or are modified by protein transfer to express one or more GPI-anchored immunostimulatory or costimulatory molecules. The tumor therapeutic composition or tumor vaccine can be prepared by a method that comprises (1) obtaining one or more GPI-anchored immunostimulatory or costimulatory molecules, and (2) transferring the GPI-anchored immunostimulatory or costimulatory molecules onto a tumor cell or isolated tumor cell membranes by protein transfer.

In another embodiment, the tumor therapeutic composition or tumor vaccine comprises (1) a microparticle encapsulating tumor antigens or peptides and (2) one or more GPI-anchored immunostimulatory or costimulatory molecules expressed on the surface of the microparticle. The tumor therapeutic composition or tumor vaccine can be prepared by a method that comprises (1) obtaining one or more GPI-anchored immunostimulatory or costimulatory molecules, and (2) transferring the GPI-anchored immunostimulatory or costimulatory molecules onto a microparticle encapsulating at least one tumor antigen or peptide, tumor lysate, tumor membranes, or combinations thereof by protein transfer.

The microparticles can be formed of any biocompatible polymer capable of incorporating GPI-anchored immunostimulatory or costimulatory molecules. For example, representative useful biocompatible polymers include, but are not limited to, polyvinyl alcohols, polyvinyl ethers, polyamides, polyvinyl esters, polyvinylpyrrolidone, polyglycolides, polyurethanes, allyl celluloses, cellulose esters, hydroxypropyl derivatives of celluloses and cellulose esters, preformed polymers of poly alkyl acrylates, polyethylene, polystyrene, polyactic acid, polyglycolic acid, poly(lactide-co-glycolide), polycaprolactones, polybutyric acids, polyvaleric acid and copolymers thereof, alginates, chitosans, gelatin, albumin, zein and combinations thereof.

The tumor antigens or peptides include, but is not limited to, mutated p53, antigenic peptides derived from p53, melanoma specific tumor antigens such as MAGE family proteins (eg MAGE-1) and peptides (eg AARAVFLAL) derived from these proteins, and combinations thereof.

GPI-anchored immunostimulatory or costimulatory molecules can be obtained by (1) expressing the GPI-anchored immunostimulatory or costimulatory molecules in a cell, and (2) isolating the GPI-anchored immunostimulatory or costimulatory molecules.

The GPI-anchored immunostimulatory or costimulatory molecules can be any substance that stimulates or costimulates immune reaction against a tumor cell that is capable of being expressed in a cell. For example, the immunostimulatory or costimulatory molecules useful here can be a cytokine molecule. In one embodiment, a useful cytokine can be, for example, one or more of cytokines IL-2, IL-4, IL-6, IL-12, CD40L, IL-15, IL-18, IL-19, granulocyte-macrophage colony stimulating factor (GM-CSF), and combinations thereof. In another embodiment, the immunostimulatory or costimulatory molecules can be, for example, the immunostimulatory or costimulatory molecules useful here can be a cytokine molecule. In another embodiment, the immunostimulatory or costimulatory molecules useful here can be, for example, B7-1, B7-2 and an intercellular adhesion molecule such as ICAM-1, ICAM-2, and ICAM-3.

The immunostimulartory or costimulatory molecules can be used alone or together and can be used in conjunction with antibody fusion proteins.

The tumor therapeutic composition or tumor vaccine described herein can be used therapeutically or prophylactically for the treatment or prevention of a tumor. Representative tumors can be treated or prevented include, but are not limited to, breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

In one embodiment, the vaccine or therapeutic composition described herein can be GPI-anchored cytokine such as GPI-IL-2 and GPI-IL-12 alone or in combination with GPI-chancored costimulatory molecules such as GPI-B7-1, GPI-B7-2, GPI-ICAM-1, GPI-ICAM-2 and GPI-ICAM-3. Such a vaccine or therapeutic composition can be used for the treatment of tumor and other diseases such as viral, bacterial and parasitic diseases.

In another embodiment, the vaccine and therapeutic composition can be biocompatible microparticles such as biodegradable microparticles modified with GPI-anchored immunostimulatory molecules such as IL-2, IL-4, IL-6, IL-12, ICAM-1, ICAM-2, ICAM-3, B7-1, B7-2, CD40L, IL-15, IL-18, IL-19, granulocyte-macrophage colony stimulating factor (GM-CSF), and combinations thereof.

In yet another embodiment, the vaccine or therapeutic compositions described herein can be tumor cells or membranes modified by protein transfer with GPI-anchored cytokines alone or/and in combination with other cytokines or/and other costimulatory molecules. One such embodiment can be, for example, tumor membranes modified with purified GPI-IL-12.

In a further embodiment, particles like inactivated or partially attenuated Virus, bacteria and virus-like particles can be modified to express immunostimulatory molecules by protein transfer with GPI-anchored cytokines and immunostimulatory molecules. Vaccines and therapeutic compositions prepared in this manner can be used for preventing or treating viral, bacterial, or parasitic diseases or disorders.

In some other embodiments, the vaccine and therapeutic compositions described herein can be used for treating autoimmune disorders. For example, membrane anchored cytokines such as IL-10 and TGF-beta can also be used to induce tolerance or to suppress immunity which can be used in treating autoimmune diseases and transplant rejection.

### BRIEF DESCRIPTION OF THE FIGURES

FIG. 1 shows a plausible mechanism for stimulation of T cell proliferation by modified tumor membranes. A) Membrane-bound immunostimulatory molecules can indirectly stimulate T cell production. B7.1 can bind to CD28 expressing mast cells and NK cells. After binding, these mast and NK cells release IFN-γ and TNF-alpha which stimulate the DCs, resulting in further T cell proliferation. Cytokines can also induce T cell differentiation through DC stimulation. In addition, B) membrane-bound cytokines and adhesion molecules can directly stimulate T cell proliferation.

FIG. 2 illustrates attaching a GPI-anchor to secreted cytokines. GPI-anchor attachment sequence and cytokine gene are recombinantly linked to form a GPI-modified cytokine that will be anchored to the cell membrane.

FIG. 3A shows modifying transmembrane proteins to have a GPI anchor; and FIG. 3B shows modifying secreted cytokines to have a GPI anchor.

FIG. 4 shows an example of modification of isolated cell membranes by protein transfer.

FIG. 5 shows some exemplary conditions of GPI-B7-1 incorporation onto isolated-tumor membranes: A) effect of temperature, B) kinetics of protein transfer, and C) effect of GPI-B7-1 concentration. Membranes were incubated with purified GPI-B7-1 at conditions specified. In all assays expression of GPI-B7-1 and MUC class I expression were determined by ELISA using specific mAbs. The level of B7-1 expression is shown relative to endogenous MHC class I expression which was designated as 1.0.

FIG. 6 shows stability of GPI-B7-1 expression on isolated-tumor cell membranes. The GPI-B7-1-modified-membranes in RPMI/2% FBS were incubated in CO₂ incubator at 37° C. Aliquots were taken at different time point and expression was determined by ELISA. The expression at day 0 was taken as 100%.

FIG. 7 shows that GPI-B7-1 modified EG7 membranes induce a tumor specific T cell immune response. Mice immunized (ip) with EG7 or GPI-B7-1 modified EG7 membranes or HBSS. After 2 weeks a booster immunization was given. Three weeks later a MTLR assay was performed to determine the tumor specific T cell proliferative response.

FIG. 8A shows that GPI-B7-1 modified EG7 membranes induces CTL activity; FIG. 8B shows that IL-12 enhances the CTL activity induced by GPI-B7-1-modified EG7 membranes; and

FIG. 8C shows depletion of CD8⁺ cells abrogates CIL activity induced by GPI-B7-1 modified EG7 membranes. Mice were immunized with indicated reagents. CTL assays were done using ⁵¹Cr-labeled EG7 and T-cells as targets and effector cells, respectively.

FIG. 9 shows that GPI-B7-1 modified EG7 membranes induced complete protection in thymoma model. Mice were immunized twice with the indicated reagents. One week after the final immunization, mice were challenged with live EG7 cells.

FIG. 10 shows GPI-B7-1 modified membranes induce partial protection in melanoma (A) and breast cancer (B) models. Mice were immunized with indicated membrane preparations. The immunization protocol is the same as used for EG7 system. After the final immunization mice were challenged with live K1735M2 (melanoma) or 4TO7 (breast cancer) cells. The membranes modified with GPI-B7-1 by protein transfer are indicated as PT-GPI-B7-1. GT-TM or GT-GPI-B7-1 indicates the membranes prepared from transfectants expressing transmembrane or GPI-anchored B7-1, respectively.

FIG. 11 shows SDS-PAGE analysis of GPI-ICAM-1 purified from CHO-cell transfectants. GPI-ICAM-1 was purified from cell lysates using anti-mouse ICAM-1 mAb-Sepharose. The eluted fractions were analyzed on SDS-PAGE followed by silver staining.

FIG. 12 shows Simultaneous protein transfer of two GPI-linked proteins. EG7 membranes were incubated with GPI-B7-1 and/or GPI-B7-1. The expressions of B7-1 (A) and ICAM-1 (B) were determined by ELISA using specific mAbs.

FIG. 13 shows flow cytometric analysis of CHO-GPI-cytokines transfectants. A. CHO cells expressing GPI-GM-CSF and GPI-IL-12 were stained with respective specific mAbs (filled histogram) or non-specific rat IgG (open histogram) and analyzed in FACScan flow cytometer.

FIG. 14 shows membrane expressed GPI-GM-CSF induce bone marrow cell proliferation. Membranes were prepared from CHO-GM-CSF and CHO cells. Membranes were incubated with bone marrow cells for 3 days. The proliferation of the cells were determined by the [³H]-thymidine uptake. Soluble GM-CSF and CHO membranes were used as controls.

FIG. 15 shows protein transfer of purified GPI-B7-1 onto microparticles. MP was incubated with PBS (buffer) or GPI-B7-1 for 20 min. The binding of B7-1 onto the MP was quantitated by ELISA using anti-B7-1 mAb (closed bar). A non-binding mIgG1, X63 (open bar) was used as control.

FIG. 16 shows GPI-B7-1 binding to the MP is through the GPI-lipid moiety: A) pretreatment of purified GPI-B7-1 with PIPLC abolishes the binding to MP, B) GPI-B7-1 bound to MP was completely released by PIPLC treatment of GPI-B7-1 modified MP, and C) soluble BSA inhibits the binding of GPI-B7-1 onto MP.

FIG. 17 shows that GPI-B7-1 bound to MP can elicit immune response. GPI-B7-1 modified MP retains its functional ability to bind to its ligand CTLA4-Ig. Binding of CTLA4-Ig was determined by ELISA using HRP-conjugated donkey anti-human IgG as detecting antibody. Unmodified MP and human IgG was used as controls.

FIG. 18 shows chimeric recombinant IL-12-CD59 is expressed as GPI-anchored form. FIG. 18A shows a schematic of the strategy to construct chimeric GPI-IL-12; and FIG. 18B shows flow cytometric analysis of P815-GPI-IL-12 cells.

FIG. 19 shows cell surface expressed GPI-IL-12 stimulates T cell proliferation. FIG. 19A shows GPI-IL-12 expressed on mastocytoma cells induces proliferation of PHA-activated human T cells; FIG. 19B shows GPI-IL-12 induces proliferation of ConA-activated mouse splenocytes; and FIG. 19C shows proliferation of activated T-cells is mediated by the cell surface expressed GPI-IL-12.

FIG. 20 shows GPI-IL-12 induces IFN-γ release by T cells. FIG. 20A shows GPI-IL-12 induces the release of IFN-γ by ConA-activated splenocytes; and FIG. 20B shows GPI-IL-12 induces the release of IFN-γ by allogeneic splenocytes

FIG. 21 shows induction of antitumor immune response by GPI-IL-12. DBA/2 mice (5-10/group) were inoculated s.c. in the right flank with 5×10⁵ live P815 (open circle) or uncloned P815-GPI-IL-12 (closed circle) or cloned P815-GPI-IL-12 (open square) or P815-secIL-12 cells (star). The mice were monitored for tumor incidence (FIG. 21A) and the tumor size (FIG. 21B) after tumor inoculation, as described under methods.

FIG. 22 shows the growth (mean tumor size) of wild type 4T07 murine breast cancer cells in groups of mice vaccinated with membranes isolated from transfected tumor cells.

### DETAILED DESCRIPTION

It has been shown that costimulatory molecules such as B7.1 can be inserted and expressed on the cell surface via a novel method of direct protein transfer (see, for example, McHugh R S, et al., Proc Natl Acad Sci USA 92:8059-8063 (1995)). The proteins are recombinantly linked to GPI lipid molecule tails, which can spontaneously insert into amphiphilic structures, such as a cell membrane (Selvaraj P, et al., Texas.: Landes Biosciences 197-211 (1999)). Studies have since optimized conditions for the incorporation of GPI-anchored proteins onto the cell surface (see, for example, Nagarajan S, et al., J Immunol Methods 184:241-251 (1995)), and purified GPI-anchored molecules are able to incorporate into the cell membrane in just 2 hours at 37 degrees Celsius (see, for example, McHugh R S, et al., Proc Natl Acad Sci USA 92:8059-8063 (1995)). GPI-linked molecules can incorporate into nucleated cells (Zhang F, et al., Proc Natl Acad Sci USA 89:5231-5235 (1992)), non-nucleated cells (Medof M E, et al., J Exp Med 160:1558-1563 (1984)), and various types of tumors, including primary breast carcinoma (McHugh R S, et al., Proc Natl Acad Sci USA 92:8059-8063 (1995)). Notably, all the studies showed that the preparation and incorporation of the GPI-linked proteins does not affect the proteins' ligand binding abilities (see, for example, Diamond D C, et al., Proc Natl Acad Sci USA 87:5001-5005 (1990)). Thus, one can quickly express immunostimulatory molecules on tumor cells by this method without the use of time-consuming gene transfer techniques for cancer vaccine development (see, for example, Ferguson M A J, et al., Annu Rev Biochem 57:285-320 (1988)). It has demonstrated that human melanoma tumor cells (SKMEL28) expressing GPI-linked B7.1 from protein transfer are able to induce an allogeneic T-cell response in vitro (McHugh R S, et al., Proc Natl Acad Sci USA 92:8059-8063 (1995)). In subsequent protein transfer studies, immunization of mice with other tagged or tailed immunostimulatory molecules, such as B7.1 and CD40 (van Broekhoven C L, et al., J Immunol 164:2433-2443 (2000)) or toxic shock syndrome toxin-1 (Wahlsten J L, et al., J Immunol 161:6761-6767 (1998)), has also been shown to initiate demonstrable antitumor responses in vivo.

Costimulatory molecules can be transferred to isolated tumor cell membranes by protein transfer. Protein transfer of costimulatory molecules to whole tumor cells has provided tumor vaccines that initiate promising antitumor immunity (see, for example, McHugh R S, et al., Proc Natl Acad Sci USA 92:8059-8063 (1995)). However, this method has various limitations, as it is difficult to establish and maintain tumor cell lines from many primary tumors, and the tumor lines that are established gradually lose the GPI-linked proteins with progressive cell divisions (see, for example, McHugh R S, et al., Proc Natl Acad Sci USA 92:8059-8063 (1995)). Additionally, the administration of live tumor cells to patients is improbable, and irradiation of cells may not be complete and may yield cells that are incapable of immunostimulation (see, for example, Chen L, et al., Cancer Res 54:5420-5423 (1994)).

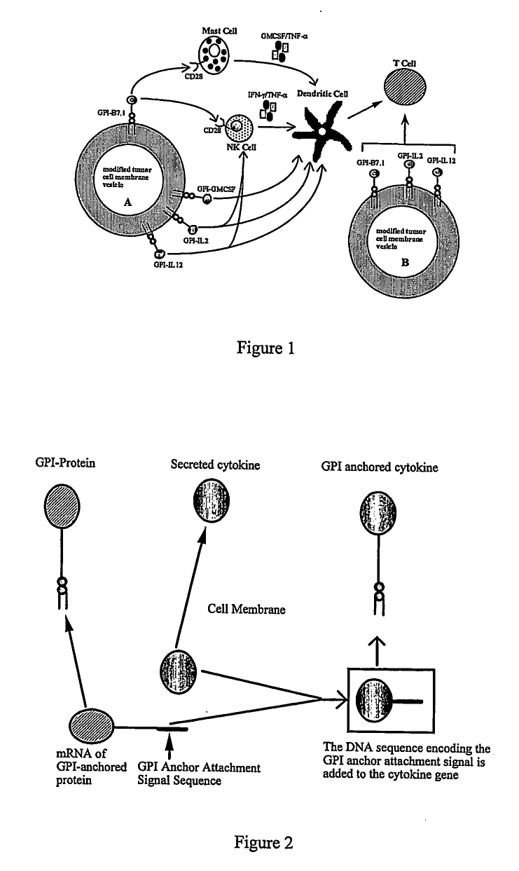

As an alternative method, it has been demonstrated that protein transfer can be used to express GPI-linked immunostimulatory molecules in preparations of isolated tumor cell membranes alone (see, e.g., Poloso N, et al., Vaccine 19:2029-2038 (2001)). B7,1-expressing membranes are effective in stimulating tumor specific T-cell and CTL proliferation and providing complete immunity to parental tumor challenge with murine T-cell lymphoma (McHugh R S, et al., Cancer Res 1999; 59:2433-2437 (1999)). Additionally, it has been shown that the cell membranes isolated from surgically removed human melanoma and renal cell carcinoma tumor tissue can be modified to express GPI-linked B7.1 by protein transfer (Poloso N, et al., Vaccine 19:2029-2038 (2001)). These membranes are able to stimulate allogeneic T cells in vitro. A plausible mechanism is that the B7.1 molecules may be acting to directly prime T cells or to indirectly prime them through interactions with other CD28 expressing cells, such as NK cells and mast cells (FIG. 1). These cells in turn can stimulate the potent DCs to process and present antigens more efficiently to T cells.

Protein transfer to tumor cell membranes, as opposed to live tumor cells, offers several advantages. Membranes do not divide or actively metabolize, thus eliminating the loss of GPI-linked molecules through cell divisions, and GPI-linked B7.1 is stably expressed for at least 7 days. Membranes prepared from patients' tumor cells can be frozen in aliquots for at least 2 years and later modified to express the GPI-linked immunostimulatory molecules for immunization (see, e.g., Poloso N, et al., Vaccine 19:2029-2038 (2001)). Additionally, the membranes already modified to express the costimulatory molecules can also be frozen and thawed with little loss of expression (see, e.g., Poloso N, et al., Vaccine 19:2029-2038 (2001)). Notably, membranes prepared from surgically removed tumor samples expressed both MHC class I and class II molecules (see, e.g., Poloso N, et al., Vaccine 19:2029-2038 (2001)), thus indicating that their use in a vaccine could possibly stimulate both CD8+ and CD4+ T cell proliferation, which would augment the antitumor response (see, Pardoll D M and Topalian S L, Curr Opin Immunol 1998; 10:588-594 (1998) (Review).

It has been recently shown that the expression of GPI-linked IL-12 molecules on tumor cell membranes (FIG. 2) induces T cell proliferation and interferon-gamma production, as well as tumor immunity in a highly tumorigenic murine mastocytoma model (Nagarajan S and Selvaraj P., Cancer Res 62:2869-2874 (2002)). Immunized mice are protected for up to 55 days from tumor challenge. A secondary advantage of GPI-linked cytokine molecules may be the creation of an insoluble slow-release depot at the vaccination site, as opposed to a transient soluble cytokine depot. A major advantage of local administration is the lack of toxicity associated with systemic administration. GPI-linked cytokine molecules can also be used in protein transfer, allowing for a more rapid preparation of cancer vaccines (see, e.g., Poloso N, et al., Vaccine 19:2029-2038 (2001)). Finally, the presence of cytokines at the site of immunization will attract cells of the immune system, increasing the rate of antigen uptake and presentation, and thus increasing the efficacy of the tumor vaccine. The GPI-linkage of the cytokine GM-CSF to the cell membrane has been engineered (Poloso N J, et al., Molecular Immunol 2002; 38:803-816 (2002)). GM-CSF stimulates DCs, key initiators of the adaptive immune response (Banchereau J and Steinman R M, Nature 392:245-252 (1998)), and potently induces antitumor immune activity (see, e.g., Hung K, et al., J Exp Med 188:2357-2368 (1998)). Studies have shown that GPI-linked GM-CSF can stimulate bone marrow cell proliferation in vitro and can induce DC generation in vivo, thus maintaining stimulatory function while anchored to the cell membrane. Additionally, the GM-CSF molecules are partially shed from the cell membrane, likely through proteolytic cleavage, resulting in local cytokine release (Poloso N J, et al., Molecular Immunol 38:803-816 (2002)). This local cytokine release promotes the migration of APCs, such as DCs, to the site of vaccination, thus facilitating tumor-specific antigen uptake and presentation.

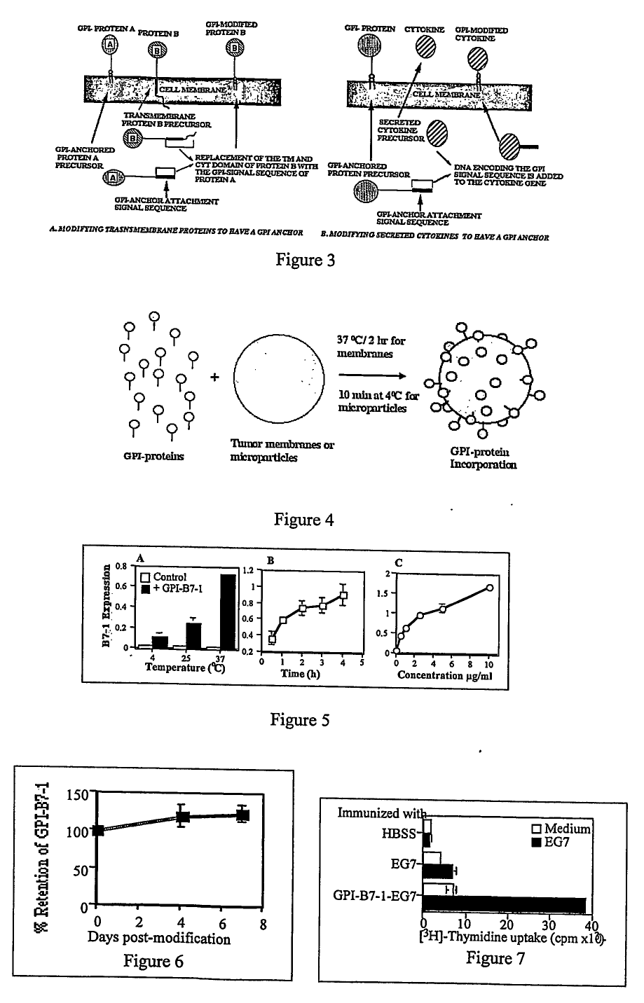

Accordingly, in some embodiments, described herein is a method of tumor treatment or tumor vaccination. The method generally comprises applying to a human being in need thereof a tumor therapeutic composition or tumor vaccine defined herein. The tumor therapeutic composition or tumor vaccine can be produced by protein transfer of glycosyl-phosphatidylinositol (GPI)-anchored immunostimulatory or costimulatory molecules (FIGS. 3 and 4). In one embodiment, the tumor therapeutic composition or tumor vaccine comprises a live tumor cell or tumor cell membranes that is or are modified by protein transfer to express one or more GPI-anchored immunostimulatory or costimulatory molecules. The tumor therapeutic composition or tumor vaccine can be prepared by a method that comprises (1) obtaining one or more GPI-anchored immunostimulatory or costimulatory molecules, and (2) transferring the GPI-anchored immunostimulatory or costimulatory molecules onto a tumor cell or isolated tumor cell membranes by protein transfer.

In another embodiment, the tumor therapeutic composition or tumor vaccine comprises (1) a microparticle encapsulating tumor antigens or peptides and (2) one or more GPI-anchored immunostimulatory or costimulatory molecules expressed on the surface of the microparticle. The tumor therapeutic composition or tumor vaccine can be prepared by a method that comprises (1) obtaining one or more GPI-anchored immunostimulatory or costimulatory molecules, and (2) transferring the GPI-anchored immunostimulatory or costimulatory molecules onto a microparticle encapsulating at least one tumor antigen or peptide, tumor lysate, tumor membranes, or combinations thereof by protein transfer.

The microparticles can be formed of any biocompatible polymer capable of incorporating GPI-anchored immunostimulatory or costimulatory molecules. For example, representative useful biocompatible polymers include, but are not limited to, polyvinyl alcohols, polyvinyl ethers, polyamides, polyvinyl esters, polyvinylpyrrolidone, polyglycolides, polyurethanes, alkyl celluloses, cellulose esters, hydroxypropyl derivatives of celluloses and cellulose esters, preformed polymers of poly alkyl acrylates, polyethylene, polystyrene, polyactic acid, polyglycolic acid, poly(lactide-co-glycolide), polycaprolactones, polybutyric acids, polyvaleric acid and copolymers thereof, alginates, chitosans, gelatin, albumin, zein and combinations thereof.

The tumor antigens or peptides include, but is not limited to, mutated p53, antigenic peptides derived from p53, melanoma specific tumor antigens such as MAGE family proteins (eg MAGE-1) and peptides (eg AARAVFLAL) derived from these proteins, and combinations thereof.

GPI-anchored immunostimulatory or costimulatory molecules can be obtained by (1) expressing the GPI-anchored immunostimulatory or costimulatory molecules in a cell, and (2) isolating the GPI-anchored immunostimulatory or costimulatory molecules.

The GPI-anchored immunostimulatory or costimulatory molecules can be any substance that stimulates or costimulates immune reaction against a tumor cell that is capable of being expressed in a cell. For example, the immunostimulatory or costimulatory molecules useful here can be a cytokine molecule. In one embodiment, a useful cytokine can be, for example, one or more of cytokines IL-2, IL-4, IL-6, IL-12, CD40L, IL-15, IL-18, IL-19, granulocyte-macrophage colony stimulating factor (GM-CSF), and combinations thereof. In another embodiment, the immunostimulatory or costimulatory molecules can be, for example, the immunostimulatory or costimulatory molecules useful here can be a cytokine molecule. In another embodiment, the immunostimulatory or costimulatory molecules useful here can be, for example, B7-1, B7-2 and an intercellular adhesion molecule such as ICAM-1, ICAM-2, and ICAM-3.

The immunostimulatory or costimulatory molecules can be used alone or together and can be used in conjunction with antibody fusion proteins.

The tumor therapeutic composition or tumor vaccine described herein can be used therapeutically or prophylactically for the treatment or prevention of a tumor. Representative tumors can be treated or prevented include, but are not limited to, breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

In one embodiment, the vaccine or therapeutic composition described herein can be GPI-anchored cytokine such as GPI-IL-2 and GPI-IL-12 alone or in combination with GPI-chancored costimulatory molecules such as GPI-B7-1, GPI-B7-2, GPI-ICAM-1, GPI-ICAM-2 and GPI-ICAM-3. Such a vaccine or therapeutic composition can be used for the treatment of tumor and other diseases such as viral, bacterial and parasitic diseases.

In another embodiment, the vaccine and therapeutic composition can be biocompatible microparticles such as biodegradable microparticles modified with GPI-anchored immunostimulatory molecules such as IL-2, IL-4, IL-6, IL-12, ICAM-1, ICAM-2, ICAM-3, B7-1, B7-2, CD40L, IL-15, IL-18, IL-19, granulocyte-macrophage colony stimulating factor (GM-CSF), and combinations thereof.

In yet another embodiment, the vaccine or therapeutic compositions described herein can be tumor cells or membranes modified by protein transfer with GPI-anchored cytokines alone or/and in combination with other cytokines or/and other costimulatory molecules. One such embodiment can be, for example, tumor membranes modified with purified GPI-IL-12.

In a further embodiment, particles like inactivated or partially attenuated Virus, bacteria and virus-like particles can be modified to express immunostimulatory molecules by protein transfer with GPI-anchored cytokines and immunostimulatory molecules. Vaccines and therapeutic compositions prepared in this manner can be used for preventing or treating viral, bacterial, or parasitic diseases or disorders.

In some other embodiments, the vaccine and therapeutic compositions described herein can be used for treating autoimmune disorders. For example, membrane anchored cytokines such as IL-10 and TGF-beta can also be used to induce tolerance or to suppress immunity which can be used in treating autoimmune diseases and transplant rejection.

The following describes some embodiments and examples of the present invention.

### Tumor Membranes Modified with GPI-B7-1 in Inducing Regression of Tumor and Memory Response

Studies have shown that that tumor immunity induced by B7-1 expressing tumors can be augmented by co-expression of other adhesion molecules, especially ICAM-1. Co-expression of B7-1 and ICAM-1 has been shown to augment anti-tumor immune responses and prolong the memory responses (Cavallo, F., et al., Eur. J. Immunol. 25:1154 (1995)). Therefore mice can be immunized with membranes modified with GPI-B7-1 and GPI-ICAM-1. Alternately, since the tumor immunity induced by B7-1 transduced tumors can be enhanced by co-administration of cytokine molecules such as IL-12 and GM-CSF, these cytokines can be co-administered during immunization.

In one embodiment, the tumor membranes described herein can be used to induce regression of established tumors. For example, tumor regression can be induced by immunization with: 1) tumor membranes modified with GPI-B7-1, 2) tumor membranes modified by GPI-B7 and co-administered with a water soluble cytokine such as IL-12 during vaccination, or 3) tumor membranes modified with combination of costimulatory molecules such as GPI-B7-1 and GPI-ICAM-1 administered with or without soluble IL-12 during immunization. Coadministration of 112 during vaccination with GPI-B7-1 modified membranes enhances CTL activity. GM-CSF can also be co-administered. Live wild type tumors cells (1×10⁶) can be injected in, for example, the left flank of the mice. The immunizations can be started on, for example, days 0, 2, 4, 6 and 9 after tumor inoculation (for a total of 5 different groups). Mice can be vaccinated with the modified membranes in, for example, the right flank. The tumor size and also the day of appearance of tumor can then be determined. At least two different vaccination regimens can be employed. A weekly injection can be used in one set of experiments, whereas a more vigorous 2 day interval immunization schedule can be used in another set of experiments to increase the antigen load further.

The effect of the tumor membranes described above can be monitored via monitoring the CD8⁺ T cell response using tetramer technology and CTL assays as described below.

Determine the Longevity of Immune Response Induced by GPI-B7-1 Modified Membranes

In every vaccination protocol it is important to determine the longevity of memory response induced by the vaccine. For example, the longevity of immune response induced by the GPI-protein modified membrane vaccine described herein can be determined.

Membranes can be prepared from EG7 tumors and modified by protein transfer to express GPI-B7-1. Groups of 10 mice can be immunized with: 1) HBSS; 2) unmodified membranes; 3) GPI-B7 modified membranes and 4) B7-1 gene transduced cells at day 0 and then challenged with 106 live, wild-type EG7 tumor cells at 2, 3, 6, 10, 20, 30, or 50 weeks later. The mice can be monitored for tumor growth. The immunization protocol can be varied by, for example, variation of dosage of membrane, frequency of immunization and membranes containing both ICAM-1 and B7-1 molecules. It has been shown that tumors expressing both B7 and ICAM-1 induce longer lasting memory response than tumors expressing either molecule alone. Accordingly, increasing the level of ICAM-1 by protein transfer of GPI-ICAM-1 can lead to efficient binding.

Tracking Antigen Specific Immune Response using H2-Kb-Tetramers and Intracellular IFN-γ Staining

In one embodiment, the kinetics and level of antigen specific CD8⁺ T cells generated can be tracked using the tetramer technology (Lee, P. P., et al., Nat. Med. 5:677 (1999); Lukacher, A. E., et al., J. Immunol. 163:3369 (1999)). The appearance of antigen specific CD8⁺ T cells in mice immunized with GPI-protein modified EG7 membranes can be followed.

Tetramers can be prepared, for example, by mixing biotinylated H2-Kb/SIINFEKL monomers with allophycocyanin-conjugated streptavidin in a 4:1 molar ratio. Mice can be immunized with tumor membranes modified with or without GPI-B7-1. As a control, activated DCs pulsed with the OVA peptide can also be used to immunize the mice. The spleen cells can be isolated at various time points after the immunization. As a control, spleen cells can be isolated from unimmunized mice. The isolated spleen cells can be co-stained with PE-conjugated anti-CD8 mAb and allophycocyanin-conjugated H2-Kb/SIINFEKL tetramer and then analyzed by flow cytometry. Analyzing the spleen cells at various time points can show the kinetics of appearance and disappearance of antigen specific CD8⁺ T cells. The level and the kinetics of appearance of the antigen specific CD8⁺ T cells in membrane immunized mice following co-administration of cytokine molecules such as IL-12 can be analyzed using this tetramer technology (Azuma, M., et al., J. Immunol 149:1115 (1992)). This tetramer technology can be used in other immunization protocols to determine the effect of coexpression of adhesion molecules, cytokine coadministration and expression on membranes, and the modified albumin MPs delivery system on the appearance and quantity of CD8⁺ antigen specific T cells.

In another embodiment, antigen specific CD8⁺ T cells can be quantitated by intracellular IFN-γ staining. The intracelluar IFN-γ staining methods have been used in measuring antiviral T cell immune responses (Lukacher, 1999, supra; Drake III, D. R., et al., J. Virol. 74(9):4093 (2000)). Unlike CTL assays, this method can quantify the number CD8⁺ T cells that functionally encountered antigen since IFN-γ is produced upon stimulation of TCR. Therefore, IFN-γ staining can be used to compliment our findings with tetramers.

As an example, spleen cells from experimental and control groups of mice can be restimulated, for example, in vitro with irradiated syngeneic spleen cells pulsed with the SIINFEKL peptide for 6 hours. As a control, spleen cells treated under similar conditions but without irradiated syngeneic spleen cells can be used. The medium can be supplemented with 1 μg/ml brefeldin A, and 50 U/ml IL-2. An aliquot of cells can be used for H2-Kb/SIINFEKL tetramer staining. Then the cells can be washed and permeabilized for intracellular staining with FITC conjugated rat anti-mouse IFN-γ mAb. The cells can also be double stained for CD8 or tetramer before flow cytometry analysis. The CD8 and IFN-γ staining can show the total number of activated antigen specific CD8⁺ T cells. Positive staining of cells with the both tetramer and anti-IFN-γ can indicate the percent activation of antigen specific T cells whereas tetramer positive but IFN-γ negative staining will show the total number of antigen specific CD8⁺ T cells.

This IFN-γ staining method along with tetramer staining can be useful in determining the kinetics and number of CD8⁺ T cells activated during the vaccination of mice with EG7 membranes. It has been demonstrated that in cancer patients nearly 2% of the CD8⁺ peripheral blood T cells can be stained with tetramers but they are negative for IFN-γ suggesting that these CTLs are not stimulated because of the persistence antigen which inactivated them (Lee, P. P., et al., Nat. Med. 5:677 (1999)).

In a further embodiment, the kinetics of generation of antigen specific T cells can also be studied in EG7 tumor system using TCR transgenic mice (OTI mice) engineered to express α and β TCR specific for H2 Kb/SIINFEKL antigen complex on their T cells (Miller, J. F., et al., Immunol. Rev. 165:267 (1998); Carbone, F. R., et al., Immunol. Today 19:368 (1998)).

### Antitumor Immunity by Tumor Membranes Modified with GPI-Anchored Cytokines by Protein Transfer

Apart from costimulatory adhesion molecules, cytokines also play a major role in the development of antitumor immune response. Accordingly, in another aspect of the present invention, tumor membranes modified with GPI-anchored cytokines can be used to induce anti-tumor immunity. The cytokines can be expressed on the cell membrane surface by the GPI-anchor. These GPI-anchored cytokines can be used to target tumor membranes to APC, such as DC, for effective antitumor immune responses.

All the cytokines can be attached to a GPI-anchor and expressed on the cell membranes (FIG. 3B). Some of these cytokines, for example, GM-CSF and IL-12, have been well investigated in many tumor systems (Jaffee, E. M., et al., Ann. NY. Acad. Sci. 886:67 (1999); Trinchieri, G. and P. Scott., Curr. Top. Microbiol. Immunol. 238:57 (1999)). Some cytokines have also been shown to synergize with B7-1 molecules when inducing antitumor immune responses (see, for example, Coughlin, C. M., et al., Cancer Res. 55:4980 (1995)). In addition, some of these cytokines, e.g., GM-CSF, can also target tumor membranes to DC cells expressing GM-CSF receptors (Kampgen, E. F., et al., J. Exp. Med. 179:1767 (1994)). Such a targeted interaction of tumor membrane may lead to increased receptor-mediated uptake of tumor membranes and simultaneous activation of DC. This can result in efficient presentation of tumor antigens by DCs and perhaps enhance MHC class I antigen presentation by cross priming.

Activation of Antitumor Immune Response by Tumor Membranes Expressing GPI-GM-CSF

As an example, tumor membranes expressing GM-CSF can be used to induce antitumor immune response. GM-CSF is known to activate dendritic cells and upregulate costimulatory molecules like B7-1. Since DC express GM-CSF receptors, GPI-GM-CSF modified membranes can interact better with DC and be taken up more efficiently than unmodified membranes. Thus, by attaching GM-CSF to tumor membranes and MPs one can simultaneously target and activate DCs, leading to efficient antigen uptake and activation of DC for effective antigen presentation. As a result, the GPI-anchored GM-CSF expressed on tumor membranes can perform the dual functions of activating DC as well as targeting tumor antigens. The following generally describes the procedures using tumor membranes expressing GPI-GM-CSF for inducing antitumor immunity.

Purify and express GPI-GM-CSF on cell membranes by protein transfer. CHO cells expressing GPI-GM-CSF can be grown in roller bottles and lysed with 1% octyl glucoside. An immunoaffinity chromatography column can be prepared using commercially available anti-GM-CSF mAbs (McHugh, R. S., et al., Proc. Natl. Acad. Sci. USA 92:8059 (1995)). GPI-GM-CSF can be purified by immunoaffinity chromatography and characterized functionally and biochemically. Tumor membranes can be modified to express GPI-GM-CSF by protein transfer and used for tumor protection.

Induction of antitumor immunity by tumor modified with GPI-GM-CSF. EG7 tumor cell line expressing GPI-anchored GM-CSF can be established by transfecting cDNA encoding GPI-GM-CSF as described in the examples. As a control EG7 cells also can be transfected with soluble GM-CSF. Mice can then be immunized with these membrane preparations. The following groups of mice can be immunized with: 1) HBSS (unimmunized control); 2) EG7 membranes modified to express GPI-B7-1 (positive control); 3) EG7 membranes expressing GPI-GM-CSF (test group); 4) Irradiated EG7 cells transfected with the secretory GM-CSF; 5) Irradiated EG7 cells expressing GPI-GM-CSF; and 6) Irradiated wild-type EG7 cells. As an additional control, DC can be isolated from Flt3 ligand injected mice (see, for example, Pulendran, B., et al., J. Exp. Med. 159:2222 (1997); Daro, E., et al., J. Immunol. 165:49 (2000)), activated with soluble GM-CSF, pulsed with SIINFEKL peptide and used to immunize. Two weeks after the immunization these mice can be challenged with EG7 tumor cells and monitored for tumor growth.

Using MHC class I tetramers and intracellular cytokine staining as described above, the antitumor immune response by tracking OVA antigen-specific CD8⁺ T cells in the above group of immunized mice can be measured. In addition to tetramer assays, the ability of GPI-GM-CSF modified and unmodified membranes to generate antigen specific CD8⁺ CTLs can be determined. Briefly, splenocytes can be harvested, for example, seven days after the last immunization and depleted of monocytes by plate adherence. The T cell enriched splenocytes can be restimulated in vitro for a period of, for example, 5 days with irradiated syngeneic spleen cells pulsed with SIINFEKL peptide. Also, following re-stimulation, the T cells can be used in a standard 4 hour ⁵¹Cr release assay to determine tumor specific CTL activity using EG7 cells as target (McHugh, R. S., et al., Cancer Res. 59:2433 (1999)). Since DC cells have receptors for GM-CSF and have the capacity to present exogenous antigens to class I pathway, the GPI-GM-CSF modified membranes can be taken up efficiently by DC and stimulate CD8⁺ T cells.

The GPI-GM-CSF modified membranes can stimulate CD4⁺ T cells since DC can present and activate CD4⁺ T cells using the MHC class II presented peptide. This can be tested using EG7 membranes modified with GPI-OVA (see the description above). As an example, mice can be immunized twice with unmodified EG membranes and EG7 membranes modified with both GPI-OVA and GPI-GM-CSF. After a period of, for example, two weeks, spleen cells can be isolated and stimulated with OVA antigen pulsed irradiated syngeneic spleen cells. T cell proliferation can be measured an assay such as by ³H thymidine incorporation assay. Before proliferation, cells in some wells can be treated with, for example, anti-CD4 mAbs and complement to deplete CD4⁺ T cells. This treatment can abolish or decrease the proliferative response, which is indicative of tumor specific CD4⁺ T cell response.

Stimulation of DC In Vitro by Tumor Membranes Modified with GPI-GM-CSF

Antitumor immunity can result from enhanced interaction of GPI-GM-CSF containing tumor membranes with DCs, which, in one embodiment, can be shown by in vitro experiments with DC isolated from bone marrow of normal mice or spleen of Flt3 treated mice. For example, the mice can be given daily injections of 20 μg of Flt3 ligand (available from Immunex) per day for ten days and the spleen cells can be isolated. DC can be isolated by for example Nycodenz gradient centrifugation. The purity and yield of cells can be analyzed by for example flow cytometry using anti-CD11c mabs. To determine the uptake of membranes, freshly isolated DC can be incubated with unmodified and GPI-GM-CSF modified EG7 membranes (for CD8⁺ T cell stimulation) or GPI-GM-CSF modified EG7 membranes further modified to express GPI-OVA antigen (for CD4⁺ T cell response). The optimum time and dose of membranes required to activate DC can be determined empirically. Then they can be evaluated for antigen presentation by measuring their ability to stimulate an OVA₂₅₇₋₂₆₄ Kb specific CD8⁺T cell hybridoma, and an I-Ab specific CD4⁺ T cell hybridoma. As controls, DC can be cultured in soluble GM-CSF, irradiated and then pulsed with OVA₂₅₇₋₂₆₄ peptide, or NP₂₀₅₋₂₁₂ (an irrelevant CTL peptide epitope derived from LCMV), or no peptide for one hour at 37° C. Peptide can then be washed out and the cells plated in for example 96 well plates in triplicate as stimulators for an OVA₂₅₇₋₂₆₄ specific CD8⁺ T cell hybridoma. As a positive control for CD4⁺ T cell hybridoma, DC can be pulsed with soluble OVA and irradiated. As a negative control, EL4 cells and DC pulsed with irrelevant peptide can be used. Co-cultures can be allowed to incubate for a period of e.g., 24 hours and the supernatants can be harvested and assayed for IL-2 release in a CTLL assay. Untreated DC will also serve as a negative control. OVA peptide pulsed DCs can be used as a positive controls for the hybridoma assays. The CD8⁺ T cell hybridoma can be stimulated to release IL-2 by the OVA peptide pulsed DCs whereas DCs pulsed with GPI-OVA modified membranes can stimulate CD4⁺ T cell hybridomas. DCs incubated with GPI-GM-CSF modified membranes can also be analyzed for expression of costimulatory molecules such as B7-1 since it has been shown that GM-CSF can induce expression of B7-1 in DC (Larsen, C. P., et al., J. Exp. Med 176:1215 (1992)). Membrane expressed GPI-GM-CSF can facilitate the uptake, which can indicate that the DC treated with membranes expressing GPI-GM-CSF can be more efficient in stimulating hybridomas than DCs treated with unmodified membranes.

In one embodiment, a combination of cytokines with adhesion molecules can be used to act in a synergistic manner in eliciting antitumor immune response. For example, tumor membranes modified by GPI-GM-CSF can be used in combinations with GPI-ICAM-1 and GPI-B7-1 in in vivo experiments such as tumor regression studies. Longevity of memory response also can be studied using this combination of molecules.

Induction of Antitumor Immunity by Tumor Membranes Modified with GPI-Anchored IL-12

As another example, cytokines such as IL-12 can be expressed in tumor membranes to induce antitumor immunity. Expression of the cytokine IL-12 has been shown to induce antitumor immunity in many tumor systems (see, for example, Chen, P. W., et al., Ann. NY. Acad. Sci. 795:325.(Abstract), 124, 125, 133-135 (1996); Zitvogel, L., et al., Eur. J. Immunol. 26:1335 (1996); Zitvogel, L., et al., Ann. NY: Acad. Sci. 795:284 (1996)). This cytokine also augments antitumor immune responses elicited by B7-1 expressing tumors (Zitvogel, L., et al., Eur. J. Immunol. 26:1335 (1996)). IL-12 is known to activate and enhance the development of antigen specific CTLs. The cytokine can also attract, inflammatory cells, NK cells, T-cells and other APCs to the vaccination site for a better immune response.

GPI-anchored IL-12 can be purified by, for example, one step immunoaffinity chromatography and express them on tumor membranes by protein transfer. Similar experimental designs and immunization protocols can be used as described above for GPI-GM-CSF, which can show that mice immunized with membranes expressing cytokines are protected from tumor challenge, which is an indication that tumor membrane modified with GPI-anchored IL-12 is capable of eliciting antitumor immune response.

GPI-anchored IL-12 can be expressed alone in tumor membranes or co-expressed with other immunostimulatory or costimulatory molecules such as ICAM-1 and B7-1 molecules to achieve synergistic effect. This membrane anchored IL-12 can also be used to cause tumor regression and memory response either alone or in combination with adhesion molecules.

### Induction of Antitumor Immunity by Tumor Antigens Encapsulated in Albumin Microparticles Modified with GPI-Anchored Molecules in Antigen Presenting Cells (APCs)

In a further aspect of the present invention, GPI-anchored immunostimulatory or costimulatory molecules can be incorporated into the surfaces of microparticles (MPs) encapsulating tumor antigens or peptides to induce antitumor immunity. For example, a modified albumin MPs delivery system can be used to target the delivery of proteins or peptides to APCs. The tumor antigens or peptides can be encapsulated in albumin MPs and then incubated with GPI-anchored proteins. Because of the hydrophobic binding pockets in the albumin, the fatty acid moiety of the GPI-anchor will bind to it. As shown in the examples, GPI-anchored proteins can be expressed on albumin MPs within 10 min. Unlike the proteins encapsulated inside the MPs, the protein transferred molecules can be expressed on the surface of the MP that makes it more accessible to interact with its cognate ligand on APCs for efficient uptake. Accordingly, by expressing appropriate proteins, MPs can be targeted to desired cells.

As shown in the examples described below, GPI-B7-1 and GPI-ICAM-1 molecules can be transferred effectively on MPs by protein transfer. Unlike protein transfer of GPI-anchored immunostimulatory or costimulatory molecules to cell membranes, the protein transfer to MP is independent of temperature, much faster, and saturation could be achieved within a relatively short period, e.g., 10 min. Studies have shown that MPs stored after freeze drying as powder retains its integrity and behaved similar to freshly prepared MPs (Willmott, N. and P. J. Harrison., Int. J. Pharm. 43:161 (1988)).

The MPs described herein can be used to present non-membranous proteins and also package more than one proteins or peptides. Therefore, this modified albumin MP system could be used for targeted delivery of any antigens for induction of an effective antitumor immune response.

Induction of Antitumor Immune Response with MPs Modified by Protein Transfer to Express GPI-B7-1, GPI-ICAM-1 and GPI-GM-CSF

In one embodiment, the effectiveness of GPI-proteins modified MPs in EG7 tumor system can be determined. Since the tumor has a known antigen, the immune response induced by encapsulated antigens, peptides, and tumor homogenate in the same tumor system can be measured. The kinetics and level of CD8⁺ T cells induced by the vaccine can be tracked using the tetramer technology. The GPI-B7-1 modified MPs, like EG7 membranes modified membranes, may not directly target and activate DC because DC do not express CD28 but it can interact with CD28 expressing NK cells and mast cells trigger inflammatory response at the site of injection. This may stimulate DC and enhance the uptake of MPs. The MPs modified with GPI-ICAM-1 and GPI-GM-CSF may directly target DC because DCs express receptors for ICAM-1 and GM-CSF.

As an example, albumin MPs can be prepared using mouse fatty acid free serum albumin as described in the preliminary studies. Desired antigens can be encapsulated during preparation of MPs and used for immunization. The EG7 tumor cells can be homogenized and centrifuged at low speed to remove the nuclei. The post-nuclear supernatant, which contains cytosolic proteins, can be encapsulated in the albumin Mps. Initially, albumin to antigen ratio can be kept at, for example, 3 to 1. In later experiments the ratio can be manipulated to increase or decrease the level of tumor antigen entrapped. Mice can be initially immunized subcutaneously with for example 20 μg of MPs per mouse with a booster shot one week later. Two weeks after the booster shot the mice can be challenged subcutaneously with live 10⁶ EG7 tumor cells. Experimental mice groups can be: 1) GPI-protein modified MPs with encapsulated tumor homogenate. 2) GPI-protein modified MPs with OVA; and 3) GPI-protein modified MPs with OVA peptides (both class I and II restricted). Protein transfer can be done with either GPI-B7-1, or GPI-ICAM-1, or both. Protein transfer with GPI-GM-CSF to MPs can be conducted by, for example, modifying MPs with immunoaffinity purified GPI-GM-CSF. Control groups can be immunized, for example, with: 1) HBSS, 2) Blank MPs, 3) MPs with OVA, 4) MPs with OVA peptides, and 5) MPs with encapsulated EG7 tumor homogenates.

The minimum and maximum immunization doses of GPI-anchored protein modified MPs can be determined. The minimum immunization dose is a maximum dose that does not result in tumor protection, which can be determined empirically. MPs modified with both GPI-B7 and GPI-ICAM-1 molecules can be more effective in inducing antitumor immune responses.

It has been demonstrated that cytokines such as IL-12 could further enhance CD8⁺ T cell expansion in EG7 tumor system (McHugh, R. S., 1999, supra). Such adjuvant effects of cytokines can be useful in further expanding CTLs, especially when the immune response is limited to a single epitope.

The antitumor T cell response in vaccinated mice can be measured using, for example, tetramer technology, in vitro T cell proliferation, and CTL assays. Tetramer staining can be carried out as described above. For CTL assays, splenocytes can be harvested, for example, 3 weeks after the last immunization, re-stimulated in vitro with EG7 cells for 5 days and tested for CTL activity by using a 4 hour ⁵¹Cr release assay against EG7 targets, using EL4 cells as non-specific controls, as described above. T cell proliferation assays on splenocytes from immunized mice in response to EG7 or EL4 tumor cells can be used to determine the anti-tumor T cell response (McHugh, R. S., 1999, supra).

Interaction with and Delivery of Antigen to DC for Presentation to T Cells by the MPs Modified with GPI-Anchored Molecules

DCs were known to express receptors for ICAM-1 (CD11a and CD11b) and GM-CSF (Kampgen, E., et al., J. Exp. Med 179:1767 (1994)). Incubation of DCs with EG7 MPs coated with ICAM-1 and GM-CSF either alone or in combination will enhance the antigen delivery. OVA encapsulated albumin MPs can be prepared and modified with GPI-ICAM-1 and GPI-GM-CSF alone or in combination. As a control, blank MPs and unmodified OVA encapsulated MPs can be used. DCs isolated from spleen cells can be incubated with these MPs for various time points and washed free of MPs. Then CD4⁺ and CD8⁺ OVA specific T cell hybridoma cells can be incubated with DC as described in specific aim 3. The supernatant can be assayed for IL-2 production. DC pretreated with GPI-GM-CSF and GPI-ICAM-1 modified MPs, can stimulate both T cell hybridomas. Since GM-CSF is a potent activator of DC, GM-CSF can be more potent than ICAM-1. Better results given by the combination of GPI-molecule modified MP can indicate that MPs modified with GM-CSF and ICAM-1 can be a better delivery system than either alone.

Application of Protein Transfer Modified Membrane and/or MP Vaccine to other Tumor Systems

In a further aspect of the present invention, GPI-B7-1 modified tumor membranes can be used to protect an animal from other tumor systems such as melanoma and breast cancer. For example, GPI-protein modified membranes are used as a vaccine to induce tumor protection in other tumor systems. In addition to B7-1, other costimulatory molecules can be used in addition to GPI-B7-1 to modify tumor membrane. Many reports have shown that the signal delivered by a combination of costimulatory molecules can synergize in stimulating T cell responses.

In one embodiment, GPI-ICAM-1 can be used in combination with GPI-b7-1. Recent studies have shown that coexpression of B7-1 and ICAM-1 augments anti-tumor immune (Cavallo, F., et al., Eur. J. Immunol. 25:1154)). Moreover, immunization with ICAM-1 transfected K1735 cells have been shown to protect mice from tumor challenge (Chen, P. W., et al., Int. J. Oncol. 6:675 (1995)), which indicating that the coexpression of ICAM-1 and B7-1 can augment the immune response induced by tumors.

In one embodiment, membranes prepared from 4T07, a murine breast cancer line, and K1735M2, a murine melanoma line, can be modified with GPI-B7-1, GPI-ICAM-1 or a combination of GPI-B7-1 and GPI-ICAM-1 by protein transfer, or left unmodified. In some experiments cytokines such as IEL-12 can be mixed with membranes before injection. Similar experimental designs, immunization protocols, and controls can be used as described above for the EG7 tumor system. As additional controls, EG7 tumor membranes modified with GPI-B7-1, K1735 and 4T07 cells transfected with either ICAM-1 and B7-1 or both can be used.

In one embodiment, tumor membranes modified with GPI-anchored cytokines can be used alone or in combination with GPI-anchored adhesion molecules to induce antitumor immunity in these tumor systems. For example, membranes prepared from 4T07 and K1735M2 can be modified with GPI-anchored cytokines by the protein transfer method. Similar procedures, vaccination protocols and controls can be used as described above for the EG7 tumors. The mechanism of action of IL-12 is different from GM-CSF. IL-12 was originally discovered as a NK cell stimulatory factor (Kobayashi, M., et al., J. Exp. Med. 170:827 (1989)). IL-12 is a chemotactic for NK cells, and also known to induce development of Th1 CD4⁺ T cells (Trinchieri, G., et al., Immunol. Today 14:335 (1993)). IL-12 gene transduced K1735M2 tumors have been shown to immunize and provide protection from tumor challenge (Coughlin, C. M., al., Cancer Res. 55:4980 (1995)). Therefore, immunization with K1735M2 tumors secreting IL-12 and expressing GPI-IL-12 can be protected from tumor challenge. Many reports have shown that GM-CSF, and IL-12 can synergize with B7-1 in inducing proliferation of CD8⁺ T cells (see, for example, Id.; Chen, P. D., 1995, supra; Stripecke, R., et al., Hum. Gene Ther. 10:2109 (1999); Bueler, H., et al., Mol. Med. 2:545 (1996)). GPI-anchored cytokines such as IL-12 can therefore be used with ICAM-1 and/or B7-1 in these tumor systems.

In a further embodiment, MWs such as albumin MPs can be used to elicit antitumor immunity in these tumor systems. The procedures and immunization of an animal can be done as described above. Unlike the EG7 system, specific tumor antigens are not available for these tumor systems and, therefore, immunization can be provided with only the MPs encapsulated with total tumor homogenates. Tumor challenge, tumor regression, and immune memory response studies can be conducted accordingly.

In these tumor systems, antigen specific CD8⁺ T cells can not be measured using the tetramers because of the lack of knowledge about tumor specific CD8⁺ T cells epitopes. The conventional CTL and T cell proliferation assays can be used to measure the cellular response in immunized animals. Alternatively, the total number of antigen specific CD8⁺ T cells can be identified by double staining for CD8 and intracellular IFN-γ after stimulation with tumor cells and used as an indication of immunity response.

### Modification of Melanoma Cells with GPI-Anchored IL-2

Construct, Express and Purin GPI-Anchored IL-2

In a further aspect of the presentation, GPI-anchored cytokines can be used to modify melanoma cells to induce antitumor immune response. In one embodiment, GPI-anchored IL-2 can be used to modify melanoma cells to induce antitumor immune response.

IL-2 is well characterized cytokine and its role in antitumor immunity is well established. mAbs and bioassays are readily available for this cytokine. In one embodiment, GPI-IL-2 cDNA can be constructed using a similar approach that was used for constructing GPI-B7 (Celis, E., et al., Molecular Immunol 31:1423 (1994)), which is described briefly below:

To create the GPI-IL-2 molecule, a DNA fragment encoding the amino acid sequence of mature secretory 12 and a CD16B DNA fragment containing a signal sequence for GPI-anchor attachment (amino acid sequences from 193 to 234) can be obtained by PCR method from cDNAs of mouse or human IL-2 (from ATCC) and CD16B, respectively. The 3′ end primer for IL-2 and 5′ end primer for CD16B will have complementary overhangs. The two PCR amplified gene fragments can be joined to form a chimeric GPI-anchored IL-2 molecule by the overlap PCR method (23). Briefly, joining of the two gene segments can be performed using an initial six cycle PCR reaction in the absence of primers. The chimera can be amplified by a second stage PCR reaction containing 0.5 μg of the IL-2 sense and CD16B antisense primers. The resulting chimera can be cloned into the shuttle vector TA (Invitrogen, San Diego, Calif.) and amplified in the DH5a strain of E. coli. The authenticity of chimeric CD16B-IL-2 cDNA construct can be verified by DNA sequencing by Sanger dideoxy sequencing method. The construct can be then subcloned into the neomycin resistant plasmid pCDNA3 (Invitrogen) using the new flanking restriction sites, XbaI and Hind III.

The chimeric gene can be ligated into the eukaryotic expression vector pCDNA3neo for transfection of CHO K1 cells (24). Unlike naturally occurring IL-2 which is secreted we expect that the GPI-anchored cytokines can be expressed on the cell surface. After selection in G418 supplemented media, the surviving cells were analyzed for IL-2 expression by FACS analysis. Further positive selection by panning can be performed to select a stable IL-2-CD16B transfectant. As a control, CHO cells either transfected with the pCDNA3neo vector alone or CD16B in CDM8 can be used.

Treatment with PIPLC can be used to confirm that the GPI-anchoring of IL-2-CD16B chimera. PIPLC is known to cleave GPI-anchored proteins expressed on the cell surface. CHO cells can be treated with 0.2 U/ml of PIPLC for 1 h at 37° C., and release of GPI anchored molecules can be analyzed by FACS. The functional activity of GPI-IL-2 can be determined by co-culturing the irradiated CHO cell transfectants with IL-2 dependent CTLL cell lines.

Alternatively, the supernatants obtained by treating CHO GPI-IL-2 cells with PIPLC can be assayed for IL-2 activity using CTLL cell line. The CHO cell transfectants can be grown in roller bottles and purified by Immunoaffinity chromatography (anti-IL-2 mAbs can be obtained from ATCC) as described (Celis, E., 1994, supra), except during column elution using octyl glucoside, a detergent which can be removed by Centricon concentrators.

Induction of Tumor Specific Immunity by the Melanoma Cells Reconstituted with GPI-IL-2

It has been shown that these melanoma cell lines induce antitumor immunity when they are transfected with IL-2 gene (Kawashima, I., et al., Cancer Res. 59:431 (1999)). K1735P class I⁺ or K1735M2 (AMC class I positive melanoma, C3H/HeN origin) transfectants secreting the IL-2 molecule can be used as a control. Mice can be immunized with tumor cells or tumor cell membrane equivalents. These tumor cells can be either control, transfected, or reconstituted with GPI-IL-2. Reconstitution of tumor cells with GPI-IL-2 can be performed as described previously (Celis, E., 1994, supra). Some mice can be repeatedly boosted with the appropriate tumor cell preparation at different intervals. After several weeks, the mice can be challenged, subcutaneously, with untreated tumor cells in 0.2 ml saline. Mice can be observed for growth of solid tumor. When a tumor of 1-2 cm in size, for example, or an ulcerated tumor has developed, the mice can be euthanized. Tumor size, as well as mouse survival, can be compared between the control and test groups for a period of, for example, up to 120 days. As a control for the effect of the reconstitution procedure, tumor membranes reconstituted with CD16B, a GPI-anchored Pc receptor, can be used as a control (Alexander, R. B., et al., Urology 51:150 (1998); Pulaski, B. A. and S. Ostrand-Rosenberg, Cancer Res. 58:1486 (1998)).

In one embodiment, tumor specific immunity can also be determined by analyzing T cells in the spleen and other lymphoid organs of control and test animal as described. For example, these lymphocyte preparations can be used to assay for CTL activity and T cell proliferation. Responder cells, which can be prepared by Histopaque isolation of lymphocytes from spleen, can be co-cultured with various amounts of irradiated stimulator cells (GPI-IL-2 positive or negative tumor cells). After several days: 1) the cells can be pulsed with 1 μCi of methyl-³H-thymidine to assay cell proliferation, or 2) the T cells can be isolated from the wells and used in a ⁵¹Cr release assay to determine CTL activity against tumor targets.

The number and dose of immunizations required for effective antitumor responses can be determined according to the procedures described herein or according to the procedures known in the art. For example, the longevity of antitumor immune response induced by tumors modified with GPI-IL-2 can be compared with that of IL-2 transfected cells to determine the efficacy of the tumors modified with GPI-IL-2 in inducing antitumor immunity.

The embodiments of the present invention will be illustrated by the following set forth examples. All parameters and data are not to be construed to unduly limit the scope of the embodiments of the invention.

### EXAMPLES

### Example 1

### Protein Transfer of GPI-Anchored Costimulatory Molecules onto Membranes Prepared from Cultured Cells and Tumor Tissue to Prepare Tumor Vaccine

Proper conditions for protein transfer. The proper conditions for the protein transfer of GPI-B7-1 onto isolated membranes were determined. Isolated-tumor membranes were prepared from tumor cells after hypotonic lysis, followed by centrifugation on a 41% sucrose solution (Maeda, T., et al., Biochim. Biophys. Acta 731:115 (1983)). GPI-B7-1 was purified from CHO cell transfectants by a single step affinity chromatography and incubated with isolated membranes. These membranes were washed and the incorporation of GPI-B7-1 was quantitated by ELISA or flow cytometry. GPI-B7-1 incorporation onto isolated-membranes was the highest at 37° C. as compared with incorporation at 25° C. and 4° C. (FIG. 5A). Another parameter shown to influence the protein transfer was the duration of incubation. As little as 30 min was enough for GPI-B7-1 incorporation, although higher level of incorporation was seen after 24 hours (FIG. 5B). Further, the incorporation of GPI-B7-1 on isolated-membranes occurred in a dose dependent manner (FIG. 5C).

Expression of GPI-B7-1 is stable under physiological conditions. A important factor in using the modified-tumor membranes as a cancer vaccine is the stability of GPI-B7-1 on isolated-membranes after protein transfer. The stability of GPI-B7-1 on the isolated-tumor membranes after protein transfer was determined. GPI-B7-1-modified-membranes in RPMI medium supplemented with serum were incubated at 37° C. Expression of GPI-B7-1 remained stable for at least 7 days at physiological temperature (FIG. 6). Stable expression of B7-1 after protein transfer was also seen in murine thymoma cell membranes. These results indicate that protein transfer of GPI-B7-1 onto isolated-tumor cell membranes can be readily accomplished.

Stability under storage conditions. In clinical settings, therapeutic isolated-membrane vaccines will most likely to be frozen for future use. To determine whether freezing isolated-membranes influenced the efficiency of protein transfer, membranes were frozen at −80° C. (from 2 days to 3 years). The efficiency of GPI-B7-1 protein transfer was tested on freshly prepared and frozen tumor cell membranes. There was no difference in the level of GPI-B7-1 incorporation onto fresh or frozen tumor cell membranes (data not shown). Moreover, endogenously expressed MEC class I expression was also not altered by freezing and thawing the membranes. This suggests that frozen membranes can be modified with GPI-anchored costimulatory molecules after storage at −80° C. for 3 years.

To see if GPI-B7-1-modified membranes could be stored for later use, GPI-B7-1-modified membranes that were stored at −80° C. for at least 2 weeks were tested. The tests showed that these GPI-B7-1-modified membranes retained 85% of GPI-B7-1 expression. In addition, membranes prepared from GPI-B7-1-transfected cells retained their ability to stimulate T cells for at least 2 years post-initial freezing at −80° C. Freezing and thawing the GPI-B7-1 modified membranes, did not affect the costimulatory function of B7-1. These results demonstrate that membranes can be stored for one or multiple immunizations.

### Example 2

### Direct Modification of Cell Membranes Isolated from Surgically Removed Tumor Tissue with GPI-Anchored Costimulatory Molecules

Establishing tumor cell lines from human tumor tissue. Out of 67 tumor samples of various histological origin, 5 cell lines could be established. This result is consistent with reports (Smythe, J. A., et al., J. Immunol. 163:3239 (1999); Simons, J. W., et al., Hum. Gene Ther. 57:1537 (1997)) that it is difficult to establish primary tumor cell lines.

GPI-B7-1 modification of tumor membranes isolated from tumor tissue. Tissues were homogenized in hypotonic lysis buffer and membranes were prepared by centrifugation on a 41% sucrose solution (Maeda, T., et al., 1983, supra). The results from several surgically removed renal cell carcinoma (RCC) and one melanoma are represented here. Flowcytometric and ELISA analysis of these membranes showed that membranes from tumor samples did not express B7-1, but express MHC class I NHC class II, and CD59. This expression of MHC class II, suggested that infiltrating leukocytes were present in the tumor tissue membrane preparation. This would be expected since macrophages and T cells do traffic to the tumor site, though most tumors do not elicit a protective immune response (Ruiz-Cabello, F., et al., Clin. Expl. Met. 7:213 (1989)). Upon further analysis of the isolated membranes, however, very low or no expression of CD2, CD3, CD4, CD8, CD16, CD32, or CD64 were detected. This finding suggests that none or very minimal membrane fragments from blood cells were present in these preparations. Alternately, it is possible that the total isolated membranes may be a mixture of the membranes from both tumor cells and low levels infiltration cells. Infiltrating macrophages or other APCs may have taken up tumor antigens. Hence, the implication of isolated membranes derived from infiltrating cells in the total membrane preparation is still significant.

Finally, to determine whether these isolated membranes from surgically removed tumor tissue could be modified by GPI-B7-1 and retain function, the isolated membranes from RCCs and melanoma tissues were incubated with GPI-B7-1 and assayed for expression. These membranes can be efficiently modified to express GPI-B7-1 by protein transfer (data not shown). These GPI-B7-1 modified membranes also bound to CTLA4 (data not shown). Most of the modified membranes could also stimulate allogeneic T cells in the presence of PMA (data not shown). These findings indicate that it is possible to isolate tumor membranes directly from tumor tissue, and could be modified with functionally active GPI-anchored costimulatory molecules by protein transfer. Thus, this method of preparing tumor membrane vaccines may obviate the need for establishing primary tumor cell lines.

Induction of tumor specific T-cell response in mice immunized with GPI-B 7-1-modified tumor membranes. The induction of antitumor immunity in vivo by GPI-B7-1-modified tumor membranes was tested in a murine thymoma model. EG7, a murine thymoma cells was used. EG7 is an ovalbumin (OVA) transfected EL4 thymoma cells with strong OVA specific CTL epitopes (Moore, M. W., et al., Cell 54:777 (1988)). Though C57BL/6 mice have OVA specific CTL, EG7 cells still form solid tumors in mice (Zhou, F., et al., Cancer Res. 52:6287 (1992)).

Induction of tumor-specific CTL. The induction of tumor specific T-cell proliferative response was determined in a mixed lymphocyte tumor cell reaction (MLTR) assay. Mice were immunized with GPI-B7-1 modified EG7 membranes. HBSS and EG7 membranes without B7-1 were used as controls. T-cells from mice immunized with GPI-B7-1-modified EG7 membranes proliferated when cocultured with irradiated EG7 cells (FIG. 7). The HBSS control and EG7 membrane primed mice were unable to mount a significant T cell proliferative response. These findings indicate that thymoma membranes modified with GPI-B7-1 induced tumor-specific T cell responses against EG7 cells.

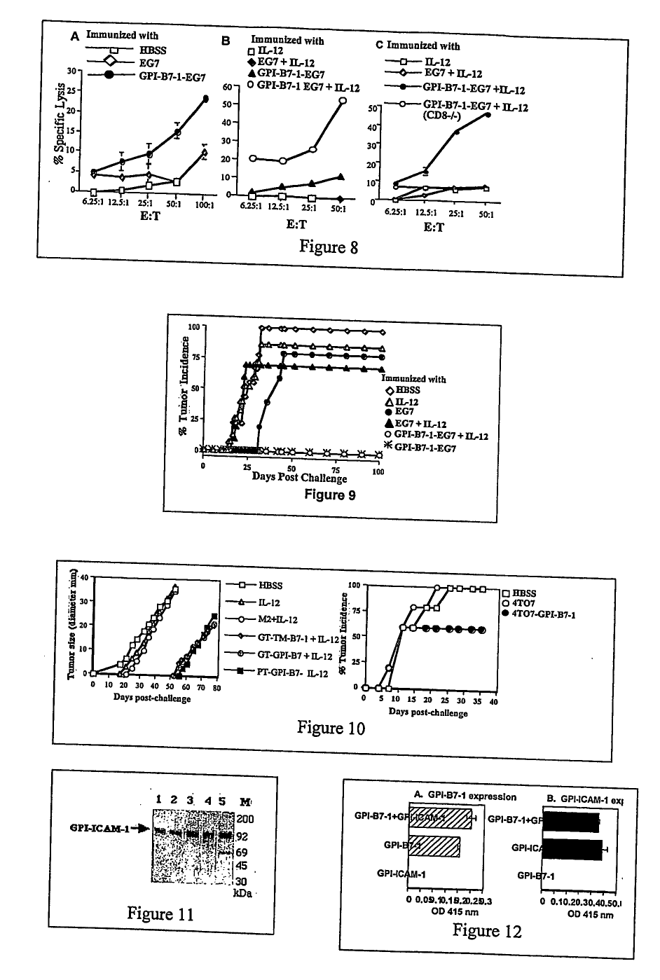

Next, a CTL response to the parental tumor was analyzed to determine whether these T-cells can kill the tumor cells in vitro. T cells from mice primed with GPI-B7-1-modified EG7 membranes had an increased cytotoxic response to the EG7 targets, compared to the T cells from mice immunized with EG7-membranes or HBSS (FIG. 8A). IL-12 has been reported to work in concert with B7-1 in generating strong CTL responses and tumor regression (see, for example, Gajewski, T. F., et al., J. Immunol. 154:5637 (1995)). Therefore, soluble IL-12 was administered during the membrane immunizations. In vivo administration of soluble IL-12 with GPI-B7-1-modified EG7 membranes and soluble IL-12 augmented CTL activity (54%) against EG7 targets (FIG. 8B). The lytic activity of the HBSS and EG7 controls remained low. These findings indicate that GPI-B7-1-modified isolated-EG7 membranes can induce CTL-specific against the EG7 tumor cells. We then determined the nature of effector cells that contributed to the specific killing of EG7 cells. Depleting CD8⁺ cells with anti-CD8 mAb and rabbit complement prior to CTL assay, reduced nearly 83% of the CTL activity (FIG. 8C), indicating that CD8⁺ cells are major effectors in the immune response to EG7 tumor cells. Similarly, in vitro depletion of CD3⁺ cells completely eliminated the cytolytic activity indicating that NK cells did not contribute to the cytotoxicity against EG7, in our CTL assays.

GPI-B7-1-modified EG7-membranes induce complete protection. After demonstrating the induction of tumor-specific Cal response, tumor protection studies were performed. Mice were immunized with GPI-B7-1 modified EG7 membranes. HBSS and EG7 membranes without GPI-B7-1 were used as controls. Two weeks after the immunization, mice were challenged with live EG7 cells. Mice immunized with GPI-B7-1-modified EG7 membranes were protected from the tumor challenge (FIG. 9). These mice remained tumor free for over 120 days post-challenge. However, in all the control groups, tumors developed after two weeks, and grew rapidly. The tumor protection studies in this thymoma model demonstrates that antitumor immunity can be induced in vivo using tumor membranes modified to express GPI-B7-1 by the protein transfer approach.

### Example 3

### GPI-B7-1-Modified Tumor Membranes Induce Partial Protection in Other Tumor Systems

The efficacy of the GPI-B7-1-modified tumor membranes to induce antitumor immunity was also evaluated in murine melanoma and breast cancer models. Membrane preparation and immunization protocols that showed complete protection in the EG7 thymoma model were used.

Delay in tumor development in murine melanoma model Membranes were prepared from K1735M2 (M2) a murine melanoma cells. These membranes were modified to express GPI-B7-1 by protein transfer. M2-transfectants expressing the transmembrane-anchored B7-1 (TM-B7-1) or GPI-B7-1 were established by transfecting corresponding cDNAs. Membranes prepared from the transfectants and wild type cells were used as controls. Tumors developed as early as 15-20 days in mice immunized with M2 membranes without B7-1 and both the control groups (HBSS or IL-12 alone) (FIG. 10A). All mice in these control groups were sacrificed because of large tumor size, before the end of this study period (80 days). Tumors did not develop until 55 days in mice immunized with GPI-B7-1-modified M2 membranes (FIG. 10A). A delay in tumor development was also seen in mice immunized with membranes from B7-1-transfected M2. These findings suggest that membranes modified with GPI-B7-1 by protein transfer and membranes from M2-B7-1-transfectants induced a weak immune response that delayed the tumor development.

Induction of partial protection in murine breast cancer model. Protein transfer studies showed that irradiated 4TO7 cells or isolated tumor membranes could be efficiently modified to express GPI-B7-1. These GPI-B7-1-modified irradiated cells and isolated-membranes were used for immunization. The immunization protocol was the same as used for the EG7 thymoma model. Two weeks after the immunization, mice were challenged with wild-type 4TO7 cells. Tumors developed in mice immunized with irradiated-cells modified with or without GPI-B7-1. However, tumors did not develop in 40% of the mice immunized with GPI-B7-1-modified membranes (FIG. 10B). These results suggest that GPI-B7-1-modified isolated tumor membranes induced a partial protection in this model. The difference between intact cells and membranes in protecting mice from tumor challenge is intriguing. It is possible that the breast cancer cells may secrete a T-cell inhibitory factor, as has been reported in the case of human breast cancer cells (107). Furthermore, the loss in expression of GPI-B7-1 incorporated onto the cells may preclude the success of enhancing immuogenicity.

### Example 4

### Construction, Expression and Characterization of Mouse GPI-ICAM-1

It has been shown that coexpression of B7-1 and ICAM-1 enhanced antitumor immune responses (Cavallo, F., et al., Eur. J. Immunol. 25:1154 (1995)). To prepare tumor vaccines expressing ICAM-1 by protein transfer, CHO cells expressing GPI-mICAM-1 by transfecting GPI-ICAM-1 cDNA were established. Transfectants expressing high level (more than 3.5 log scale) of GPI-ICAM-1 were obtained by cell sorting and panning procedures. GPI-ICAM-1 was purified from CHO-GPI-ICAM-1 cell lysates using anti-mouse ICAM-1-mAb-Sepharose column as described (54). SDS-PAGE analysis of the eluted fractions showed most of the fractions contained highly purified GPI-ICAM-1 of a molecular weight about 90 kDa (FIG. 11). Only the fractions with highest purity can be used for in vivo studies. The functional integrity of the purified GPI-ICAM-1 was determined in an inversion plate binding assay as described (McHugh, R. S., et al., Proc. Natl. Acad. Sci. USA 92:8059 (1995)). Since mouse ICAM-1 is known to bind to human LFA-1 (109), LFA-1+ human T-cell line (SKW3) was used for this assay. SKW3 cells bound to ICAM-1 coated wells (data not shown) with very minimal background binding to wells without ICAM-1. Addition of anti-mouse ICAM-1 (YN) and anti-human LFA-1 (TS1/22) mAbs completely blocked this binding, indicating that this binding is specific. These results indicate that purified GPI-ICAM-1 retains its functional activity to bind to LFA-1.

Membranes can be modified to express at least two GPI-anchored proteins by protein transfer. Using the protein transfer approach, it is possible to express more than one protein on tumor membranes, which was investigated by doing the protein transfer using GPI-B7-1 and/or GPI-ICAM-1 onto EG7-membranes. GPI-B7-1 or GPI-ICAM-1 alone incorporated efficiently onto membranes. The combined presence of both of them during protein transfer did not affect the incorporation of the other (FIG. 12). These results suggest that addition of at least two GPI-anchored proteins at the concentrations tested do not affect the efficiency of protein transfer.

### Example 5

### Construction, Expression and Characterization of GPI-Anchored Cytokines

Construction of GPI-signal sequence cassette. In order to make cDNA encoding various recombinant GPI-anchored molecules easily, a base-cassette with GPI-anchor signal sequence of CD59 with an Afl II linker at 5′ end was constructed. This cassette was constructed by cloning a truncated-B7-1-CD59 cDNA in pcDNA3^{neo} at EcoR V/Apa I sites. The strategy to clone a cDNA of a desired protein encoding GPI-anchored form includes: a) PCR amplification of the desired cDNA with Afl II linker at 3′ end with Pfu DNA polymerase (creates blunt ends), b) digesting the PCR product with Afl II, c) excising truncated B7-1 with EcoR V/Afl II that will leave the CD59 sequence with the vector backbone, and d) cloning the Afl II digested PCR product into the cassette at EcoR V/Afl II sites. Using this cassette, a cDNA encoding the GPI-anchored form of desired molecule can be readily constructed.

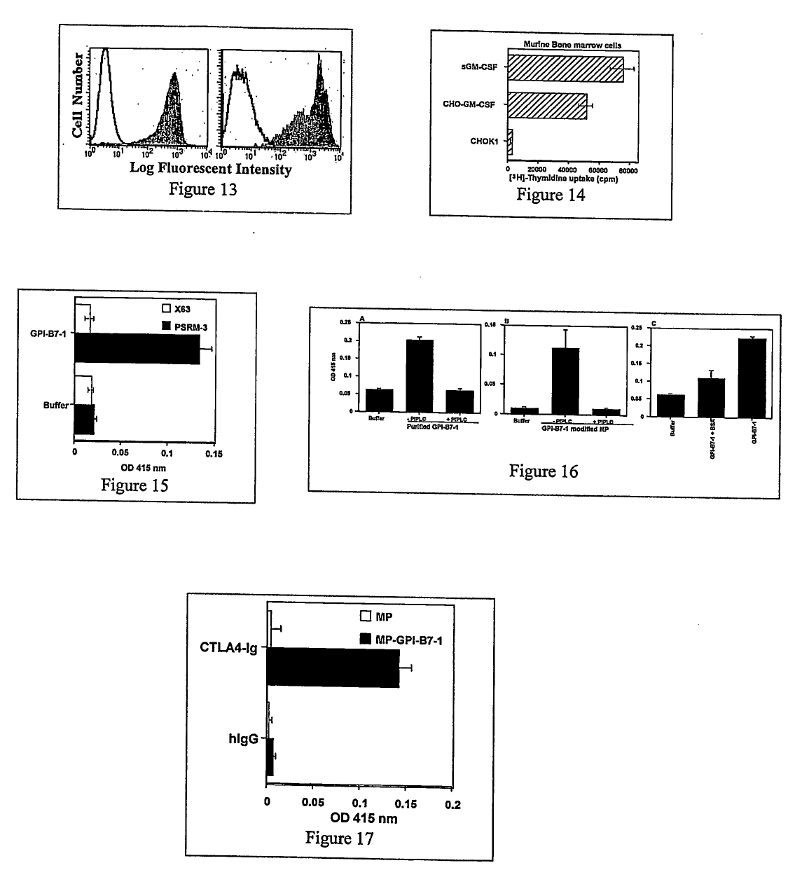

Expression of GPI-anchored murine GM-CSF and IL-12 in CHO cells. GPI-anchored GM-CSF and IL-12 were constructed using the strategy described above. The coding region of cytokines were obtained by RT-PCR and cloned into the CD59-casette. CHO transfectants expressing GPI-GM-CSF was established by transfecting the GM-CSF-CD59 cDNA. Flow cytometric analysis of these transfectants showed that GM-CSF was expressed as GPI-anchored form (FIG. 13A). PIPLC (an enzyme that cleaves the GPI-anchored proteins) treatment of CHO-GPI-GM-CSF cells showed complete release of GM-CSF from the cell surface. This finding indicates that GM-CSF is expressed on the cell surface as GPI-anchored form.

Unlike GM-CSF, IL-12 is expressed as a heterodimer consisting of 35 kDa and 40 kDa subunits. A similar strategy was used to construct the GPI-anchored forms of p35 and p40. However, to establish CHO cell transfectant expressing the heterodimeric IL-12, p35-CD59 cDNA was mobilized from pcDNA3^{neo} and cloned into pUB6^{bla} at Kpn I and Apa I sites. CHO cells transfected with p35-CD59 and p40-CD59 cDNAs were selected in blasticidin and G418. As shown in FIG. 13B, CHO-GPI-IL-12 transfectants showed the cell-surface expression of IL-12. The GPI-anchored form is confirmed by PIPLC treatment. Western blot analysis of GPI-IL-12 showed a protein band corresponding to 80 kDa under non-reducing conditions. Under reducing conditions using DTT, two bands corresponding to 35 and 40 kDa was seen (data not shown). These results indicate that the GPI-IL-12 folded correctly and was expressed as a heterodimer.

Membrane expressed GPI-cytokines are functional. The functional integrity of GPI-anchored-GM-CSF was determined in cell proliferation assay using murine bone marrow cells. The GPI-GM-CSF expressed on the CHO cells induced the proliferation of the respective responder cells. Furthermore, membranes prepared from CHO cells expressing GPI-GM-CSF also induced the proliferation of bone marrow cells (FIG. 14). These findings indicate that the membrane-expressed-GPI-GM-CSF retain their functional ability to induce cell proliferation.

### Example 6

### Modification of Albumin Microparticles with GPI-Anchored Immunostimulatory Molecules by Protein Transfer

Microparticle preparation: Albumin microparticles were prepared by a previously described modified water in oil emulsion technique (D'Souza, M. J., et al., J. Interferon and Cytokine Research 19:1125 (1999)). Briefly, bovine serum albumin in PBS was homogenized into olive oil using a bio-homogenizer for 10 minutes to form an emulsion of the microparticles. Once the microparticles were formed the surface of the microparticles were cross-linked and stabilized with glutaraldehyde and stirred for 6 h. The olive oil was then washed off with acetone followed by centrifugation to separate the microparticles. Sizing of the microparticles was done using sequential HPLC type nylon filters. The microparticles were freeze dried and stored in a refrigerator until used.

The microparticles were prepared using albumin. Albumin has hydrophobic pocket that can bind to free fatty acids. This allows an albumin-MP to bind to fatty acids moieties in GPI-anchor. Albumin-MP was incubated with purified GPI-B7-1 and the binding of B7-1 was determined by ELISA. As shown in FIG. 15, GPI-B7-1 bound to MP as this binding was detected by anti-B7-1 mAb (PSRM-3). MP incubated in buffer without GPI-B7-1 did not bind to anti-B7-1 mAb. Moreover, a non-specific mIgG (X63) did not bind to MP-modified with GPI-B7-1. These findings indicate that GPI-B7-1 specifically attached to the MP. The optimal conditions for GPI-B7-1 binding to MP were determined. The binding of GPI-B7-1 to MP was saturated as early as 10 min and this binding is independent of the incubation time up to 90 min. The levels of GPI-B7-1 attachment to MP was similar at 4° C., 22° C. and 37° C., indicating that the binding of GPI-B7-1 to MP, unlike membranes, did not depend on the incubation temperature.

The effect of GPI-B7-1 concentration on its binding to MP was investigated, and a dose-dependent increase in the binding of GPI-B7-1 to MP was observed. In addition, more than one costimulatory molecule could be attached to the MP. Addition of GPI-B7-1 and/or GPI-ICAM-1 with MP during protein transfer showed that both molecules can be incorporated efficiently onto MP alone or in combination. These findings show that the albumin-MP can be modified to express GPI-anchored costimulatory molecules. Further, the proper conditions for protein transfer of MP differ from that seen with intact cells or cell membranes (Nagarajan, S., et al., J. Immunol. Methods 184:241 (1995); McHugh, 1995, supra; McHugh, 1999, supra).

GPI-B7-1 bound to microparticles through the lipid moiety of GPI-anchor. The mechanism of this binding of GPI-B7-1 onto MP was elucidated. Earlier studies from our laboratory have shown that the incorporation of GPI-anchored proteins could be inhibited by bovine serum albumin (Nagarajan, 1995, supra). It is well established that serum albumin can bind to fatty acids. Three criteria, described in the following, were used to determine if the binding of GPI-B7-1 onto albumin-MP may be mediated through the fatty acid moieties present in the GPI-anchor: First, the presence of soluble BSA during the GPI-B7-1 and MP incubation inhibited the binding of GPI-B7-1 to MP (FIG. 16A). Secondly, pretreatment of purified GPI-B7-1 with PIPLC, completely abolished the binding of GPI-B7-1 to MP (FIG. 16B). Third, PIPLC treatment of GPI-B7-1-modified MP resulted in complete release of B7-1 from the MP (FIG. 16C). These findings indicate that the GPI-B7-1 bound to the albumin-MP through its GPI-anchor.

GPI-B7-1-modified MPs are functional. The functional integrity of GPI-B7-1 bound to MP was then determined using recombinant CTLA4-Ig. CTLA4-Ig specifically bound to GPI-B7-1-modified MP (FIG. 17). However, CTLA4-Ig did not bind to MP without B7-1. Furthermore, human IgG did not show any detectable binding to GPI-B7-1-modified MP, suggesting that CTLA4 binding is specific.

### Example 7

### Tumor Vaccine by GPI-Anchored IL-12 (GPI-IL-12)

### Materials and Methods

Cell Lines, Monoclonal Antibodies and Cytokines

Murine mastocytoma (P815), rat hybridomas against murine MHC class I (M1/42), CD54 (YN1.1), CD80 (IG10) and CD24 (1/69) were purchased from ATCC (Manassas, Va.). Rat anti-murine IL-12 hybridomas (C15.6 and C17.8) were kind gifts from Dr. Trinchieri (Wistar Institute, Philadelphia, Pa.). P815 cells were cultured in DMEM supplemented with 5% FBS, 2 mM glutamax I (Invitrogen, Carlsbad, Calif.), 1 mM sodium pyruvate, penicillin (100 units/ml), and streptomycin (100 μg/ml), 55 μM-mercaptoethanol, and gentamicin 50 μg/ml (cDMEM). The hybridomas were maintained in RPMI 1640 supplemented with 10% calf serum (Hyclone, Logan, Utah), 2 mM glutamine, and other additives at concentration mentioned above (complete RPMI). All cell culture reagents were purchased from Mediatech Inc (Hemdon, Va.), unless indicated. Unconjugated and HRP- or FITC-conjugated-F(ab)₂ goat anti-mouse IgG and F(ab′)₂ goat-anti-rat IgG were purchased Jackson Immunochemicals (West Grove, Pa.). Mouse anti-human IFN-γ mAbs (Clones 2G1 and B133.5) were purchased from Pierce Endogen (Rockford, Ill.). Rat anti-mIFN-γ mAbs (clones R4-6A2 and XMG1.2) were kind gifts from Dr. K. Ziegler (Emory University, Atlanta, Ga.).

Construction of GPI-IL-12 and Secretory IL-12 cDNAs

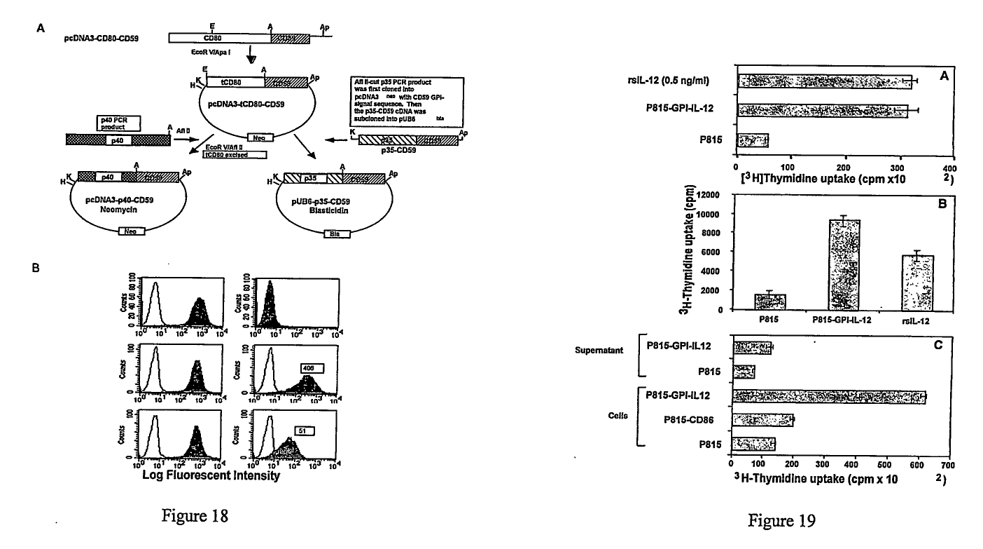

A mammalian expression vector cassette with GPI-anchor signal sequence of CD59 (containing Afl II linker at 5′ end of CD59 cDNA) was constructed by cloning a truncated-human CD80-CD59 cDNA in pcDNA3^{neo} (Invitrogen, Carlsbad, Calif.) at EcoR V/Apa I sites (FIG. 18A). This expression vector cassette was used to make cDNAs encoding the GPI-anchored form of mouse IL-12 (GPI-IL-12). IL-12 is a disulfide-linked heterodimer consisting of 35 and 40 kDa polypeptides (Trinchieri, G. and Scott, P., Curr. Top. Microbiol. Immunol., 238: 57-78, (1999)). The coding regions (excluding the stop codon) of both subunits of IL-12 were PCR amplified using pNGVL3-IL-12 cDNA as the template using Pfu DNA polymerase (Stratagene, La Jolla, Calif.). The primers to amplify p35 cDNA were, forward (catccagcagctcctctca) and reverse (cattgcttaaggcggagctcagatagccc); and the following forward (gcacatcagaccaggcagct) and reverse (ccattgcttaaggatcggacectgcagggaa) primers were used to amplify cDNA encoding p40 kDa subunit of IL-12. The reverse primers were designed to have an Afl II linker (underlined). The truncated CD80 cDNA was excised from the tCD80-CD59-pcDNA3^{neo} mammalian expression vector with EcoR V/Afl II, which leaves the GPI-anchor addition signal sequence of CD59 with the vector cassette (FIG. 18A). The Afl II-digested PCR products of p35 and p40 kDa cDNAs were then cloned into the cassette containing GPI-anchor addition signal sequence of CD59 at EcoR V/Afl II sites. Using this strategy, both p35-CD59 and p40-CD59 cDNAs were cloned into pcDNA3^{neo} mammalian expression vector (FIG. 18A), and the p35-CD59 cDNA was further subcloned into pUB6^{blasticidin} (pUB6^{bla}) vector (Invitrogen, Carlsbad, Calif.). cDNA encoding secretory IL-12 (secIL-12) was mobilized from pNGVL3-IL-12 and cloned into pUB6^{bla} (pUB6^{bla}-secIL-12) at Kpn I and Apa I sites

Establishing transfectants expressing GPI-anchored or secretory IL-12. P815 transfectants expressing GPI-IL-12 were established by transfecting murine p35-CD59-pUB6^{bla} (10 μg) and p40-CD59-pcDNA3^{neo} (10 μg) cDNAs by electroporation using a BioRad gene pulser II (Hercules, Calif.). The electroporation was performed using the cells in serum free RPMI 1640 pulsed at 960 μF and 0.25 kV/cm. After 48 h of transfection, the GPI-IL-12⁺ cells were enriched by biomagnetic selection using anti-IL-12 mAb (C17.8) and sheep anti-rat IgG magnetic beads (10 beads/cell) and two cycles of panning method as described earlier (McHugh, R. S., Proc. Natl. Acad. Sci. USA., 92: 8059-8063, (1995)). The enriched GPI-IL-12+ cells (uncloned) were cultured in cDMEM containing blasticidin (10 μg/ml) and G418 (1 mg/ml). P815 cells secreting IL-12 (P815-secIL-12) were established by transfecting pUB6^{bla}-secIL-12 cDNA. Cells secreting IL-12 was selected in cDMEM containing blasticidin (10 μg/ml). Single cell clones of P815-GPI-IL-12 and sec-IL-12 were established by limited dilution cloning. The uncloned and cloned P815-GPI-IL-12 transfectants were used in this study. To determine the cell surface expression of IL-12, MHC class I, CD54, CD80, and CD24 on uncloned and cloned populations, the cells were stained with appropriate mAbs, and analyzed using a FACScan flow cytometer (Becton-Dickinson, San Jose, Calif.). To confirm the GPI-linkage of cell surface expressed IL-12, cells were treated with phosphatidylinositol-specific phospholipase C(PIPLC) (Id.) followed by flow cytometric analysis. To determine the growth characteristics of GPI-IL-12⁺ tumor cells in vitro, P815 or P815-GPI-IL-12 cells (1×10³) were cultured in cDMEM for 24 h at 37° C. The cells were then pulsed with ³H-thymidine (1 μCi/well) and incubated for another 18 h. ³H-Thymidine uptake was determined in a Packard Top count scintillation counter.

Preparation of Isolated Membrane Vesicles from P815-GPI-IL-12 Transfectants.

Isolated membranes were prepared from P815 and P815-GPI-IL-12 cells by sucrose gradient ultracentrifugation (McHugh, R. S., Cancer Res., 59: 2433-2437 (1999); Poloso, N., et al., Vaccine., 19: 2029-2038 (2001)). Membranes were resuspended in protein free RPMI with antibiotics and frozen in aliquot at −80° C. Protein concentrations of membranes were determined by BioRad dye binding method using BSA as standard. Expression of GPI-IL-12 and other surface markers on the isolated membranes were determined by ELISA using appropriate mAbs (Id.). To quantitate IL-12 expressed on isolated membranes, GPI-IL-12⁺ isolated membranes (150 μg) were lysed in 20 mM Tris-HCl (pH 8.0) containing 1% octyl β-glucoside for 1 h and centrifuged at 20,000×g for 1 h to collect clear lysate. IL-12 in the lysate was determined by sandwich ELISA using anti-IL-12 mAbs (C17.8 and biotinylated-C15.6) and HRP-conjugated avidin. Color was developed using TMB-1 as substrate, and reaction was stopped with 2N H₂SO₄. The color developed was read at 415 nm in an ELISA microplate reader (Molecular Devices, Sunnyvale, Calif.). Isolated membranes prepared from P815 cells were treated identically and used as a negative control.

Proliferation of PHA-activated human T cells and ConA-activated murine splenocytes.

T cells were enriched from peripheral blood mononuclear cells isolated from healthy donor as described (Poloso, 2001, supra). PHA-activated human T cells were prepared using 1% PHA (Invitrogen, Carlsbad, Calif.) by standard procedure (Schoenhaut, D. S., et al., J Immunol., 148: 3433-3440. (1992)). P815 and P815-GPI-IL-12 cells (stimulators) were treated with mitomycin C (50 μg/ml) for 30 min at 37° C., washed extensively with complete RPMI and used in the proliferation assays. PHA-activated T cells (responders) were co-cultured with mitomycin C-treated stimulator cells for 72 h. Cells were pulsed with ³H-thymidine (1 μCi/well) (Amersham, Arlington Heights, Ill.) for the final 18 h and harvested using a Packard filtermate cell harvester (Meriden, Conn.). ³H-Thymidine uptake was counted in a Packard Top count microplate scintillation and luminescence counter (Downers Grove, Ill.). Similarly, proliferation of ConA-activated splenocytes (responder) was done by co-culturing responders with mitomycin C-treated stimulator cells for 72 h. The uptake of ³H-thymidine after 18 h pulse with ³H-thymidine (1 μCi/well) was determined as described above.

MLTR and IFN-γ Release Assay.

An allogeneic mixed lymphocyte tumor reaction (MLTR) assay was carried out to determine the efficacy of GPI-IL-12 to induce alloantigen specific T cell stimulation. Mitomycin C-treated P815 (H-2^{d}) or P815-GPI-IL-12 cells were co-cultured for 72 h with unactivated splenocytes of C57BL/6 (H-2) mice. Recombinant soluble murine IL-12 (rsIL-12) was included as a positive control. The MLTR cultures were centrifuged and the supernatant was analyzed for the release of IFN-γ to determine the IL-12-dependent T cell stimulation. IFN-γ release was determined by sandwich ELISA using corresponding mAb pairs. Similarly, to determine the IL-12-dependent stimulation of activated-T cells, P815-GPI-IL-12 cells or membranes isolated from P815-GPI-IL-12 cells were co-cultured with ConA-activated mouse splenocytes or PHA-activated human T cells as responders. The release of IFN-γ by activated-T cells was used as a measure to determine the IL-12-dependent T cell stimulation. Supernatants were collected after 48 h and the release of human or murine IFN-γ was determined by sandwich ELISA using paired mAbs.

Tumor Challenge Studies.

Female DBA/2 mice (6-8 weeks) were purchased from the Jackson Laboratory (Bar Harbor, Me.) and maintained in Emory University animal facility according to the regulations of institutional animal care and use committee. Mice (5-10 mice/group) were challenged (s.c.) with P815 or P815-GPI-IL-12 or P815-secIL-12 cells (5×10⁵ cells/mice), and were monitored twice a week for tumor growth. Two measurements of tumors that are perpendicular to each other were measured using vernier calipers. Tumor size (mm²) was quantitated by multiplying the two diameters for each mice in control and experimental groups. Mice were euthanized when tumor size reached >2 cm. To determine the presence of IL-12 in the systemic circulation, mice (3 per group) were injected with serum free RPMI or live P815 or P815-GPI-IL-12 or P815-secIL-12 cells (5×10⁵ cells in 200 μl). Serum samples were collected, pooled (3 mice/group) and IL-12 and IFN-γ in serum samples were quantitated by sandwich ELISA using appropriate mAbs.

### Results

The above example shows that chimeric IL-12-CD59 can be expressed on the cell surface as a GPI-anchored protein. The cDNAs encoding the entire coding region of p35 and p40 subunits of mouse IL-12 were ligated in-frame to a GPI-anchor addition signal sequence of CD59 in a mammalian expression vector cassette (FIG. 18A). Stable transfectants of a murine mastocytoma, P815, expressing mouse GPI-IL-12 was established by co-transfecting chimeric cDNAs of p35 and p40 subunits, as described under methods. Flow cytometric analysis of the P815-GPI-IL-12 transfectants showed cell surface expression of IL-12 (FIG. 18B). In addition, the expression of other cells surface markers, such as MHC class I, CD54 were not altered in this transfectants, as compared to the P815 cells (FIG. 18B). More than 90% of the GPI-IL-12 protein expressed on transfected cells was released by PIPLC treatment (FIG. 18B), indicating that the IL-12 is anchored to the cell surface via a GPI-moiety.

This example also shows that GPI-IL-12 expressed on cell surface anchored to the membrane via GPI-moiety is capable of inducing T-cell proliferation. Murine rsIL-12 has been shown to stimulate activated human and murine T cells (Schoenhaut, D. S., 1992, supra). Therefore, the functional integrity of GPI-IL-12 was determined for its ability to induce the proliferation of activated-T cells. PHA-activated human T cells were co-cultured with mitomycin C-treated P815-GPI-IL-12 cells. GPI-IL-12+ cells induced T cell proliferation and levels of proliferation were similar to that obtained using 0.5 ng/ml of rsIL-12 (FIG. 19A). Similarly, ability of GPI-IL-12⁺ cells to induce the proliferation of ConA-activated murine splenocytes was also determined. P815-GPI-IL-12 cells were able to induce proliferation of ConA-activated splenocytes, over P815 cells (FIG. 19B). It has been shown that some GPI-anchored proteins such as CD16B are released from the cell surface (Huizinga, T. W. J., et al., Nature, 333: 667-669 (1988). Therefore, to determine if the induction of T cell proliferation was mediated by the cell surface expressed GPI-IL-12, and not due to shedding or secretion of IL-12, P815 and P815-GPI-IL-12 cells were cultured in cDMEM and supernatants were collected after 48 h. Supernatants were centrifuged at 100,000×g to remove any membrane fragments or particulate materials and tested for the presence of IL-12 in a T cell proliferation assay using a PHA-activated human T cells. As shown in FIG. 19C, mitomycin C-treated P815-GPI-IL-12 induced proliferation of PHA-activated human T cells, whereas P815 or P815-CD86 cells did not. However, under the similar assay conditions, the supernatant obtained from P815-GPI-IL-12 cells did not induce proliferation, suggesting that there is no detectable level of IL-12 released into the supernatant from P815-GPI-IL-12 cells. These findings indicate that GPI-IL-12 is expressed as a functionally active heterodimer on the cell surface.

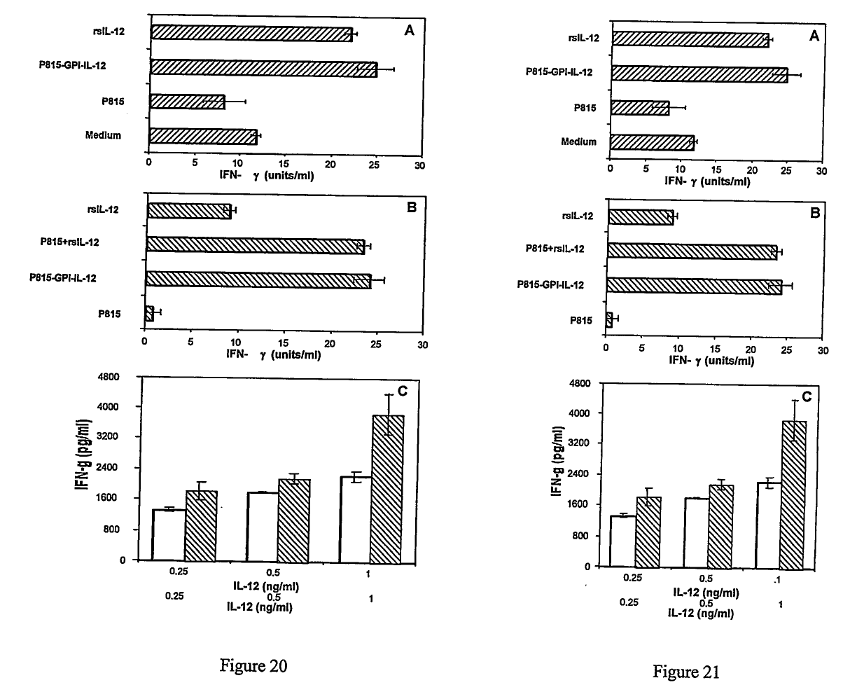

This example further shows that cell-surface expressed GPI-IL-12 is capable of inducing the release of IFN-γ by activated-splenocytes. It has been well established that IL-12 can stimulate T and NK cells and induce the release of Th1 type cytokines such as IFN-γ (Trinchieri, G. and Scott, P., Curr. Top. Microbiol. Immunol., 238: 57-78 (1999)). Therefore, the ability of the cell surface expressed GPI-IL-12 in inducing the release of IFN-γ was tested. P815 cells did not induce IFN-γ release from the activated cells. However, co-culturing P815-GPI-IL-12 cells induced IFN-γ release by ConA-activated splenocytes (FIG. 20A).

This example further shows that GPI-IL-12 is capable of augmentation of allogeneic T cell stimulation. The induction of allogeneic T cell stimulation by GPI-IL-12 was determined in a MLTR assay. Unactivated splenocytes from C57BL/6 mice (H-2^{b}) were co-cultured with mitomycin C-treated P815 (H-2^{d}) or P815-GPI-IL-12 cells. IFN-γ released by the stimulated allogeneic splenocytes was measured to determine the IL-12-dependent T cell stimulation. Addition of P815-GPI-IL-12 cells induced the release of IFN-γ as compared to P815 control (FIG. 20B). Similar levels of IFN-γ were observed when allogeneic splenocytes were co-cultured with P815 cells mixed rsIL-12. Under similar conditions very low levels of IFN-γ release by allogeneic T cells was seen in presence by rsIL-12 alone, and P815 cells did not induce release of IFN-γ. These findings indicate that the increased release of IFN-γ seen with P815-GPI-IL-12 is due to augmentation of alloantigen-mediated T cell stimulation.

This example further demonstrates that membrane vesicles isolated from P815-GPI-IL-12 cells are capable of inducing release of IFN-γ. A potential application of making GPI-anchored cytokines such as GPI-IL-12 is that the purified GPI-IL-12 can be used to modify isolated tumor membranes for vaccine preparation by protein transfer approach (McHugh, R. S., 1999, supra; Poloso, N., 2001, supra). Moreover, the isolated tumor cell membranes expressing GPI-IL-12 can also be used as a vaccine for intratumoral administration. Therefore, the isolated cell membranes were prepared from P815-GPI-IL-12 cells and determined whether it can induce stimulation of activated-T cells. The isolated membranes showed the expression of GPI-IL-12 and other surface markers such as MHC class I and CD54 (data not shown). The release of IFN-γ by ConA-activated murine splenocytes and PHA-activated human T cells were used as a measure to determine the IL-12 dependent T cell stimulation. Addition of GPI-IL-12⁺ isolated cell membranes in the proliferation assay resulted in the release of IFN-γ by ConA-activated splenocytes. The level of IFN-γ release induced by the GPI-IL-12⁺ isolated cell membranes is comparable to that seen with rsIL-12. Membranes prepared from P815 cells did not induce the release of IFN-γ from the activated cells. Similarly, membranes prepared from P815-GPI-IL-12 cells also showed increase in IFN-γ release by PHA-activated human T cells (data not shown). These findings indicate that the isolated membranes expressing GPI-IL-12 retained its functional activity to stimulate activated-T cells.

This example further demonstrates the antitumor immune response induced by GPI-IL-12 expressed on tumor cells. Prior to using the P815-GPI-IL-12 transfectants in animal studies, the growth characteristics of P815-GPI-IL-12 cells in vitro were determined in a proliferation assay as described under methods. The basal proliferation of P815 and P815-GPI-IL-12 cells were similar (data not shown), indicating that transfecting GPI-IL-12 into mastocytoma cells did not change the growth characteristics of the cells in vitro. The ability of cell surface expressed GPI-IL-12 to induce antitumor immune response in vivo was determined using a highly tumorigenic and moderately immunogenic mastocytoma tumor model. Mice were inoculated with live P815 or P815-GPI-IL-12 cells and monitored for tumor development and survival. To compare the efficiency of secretory versus GPI-anchored IL-12 in inducing an antitumor response tumor studies were done using P815 cells expressing GPI-IL-12 (uncloned cells established by panning) or cloned P815-GPI-IL-12 or P815-sec-IL-12 cells. The mice inoculated with control P815 cells developed tumors by day 10 and tumors grew progressively (FIG. 21A). All the mice in this control group were either dead or euthanized (when the tumors reached the allowed limit) after 44 days post-inoculation of P815 cells FIG. 21B). However, all the mice inoculated with uncloned P815-GPI-IL-12 cells survived and were tumor free up to 55 days (FIGS. 21A and 21B). Tumors developed only after 55 days of tumor inoculation in 40% of mice, and all the mice in this group developed tumor by day 80. Interestingly, all the mice inoculated with cloned GPI-IL-12 or secIL-12 cells were tumor free even after 75 days (FIGS. 21A and 21B). To determine whether the tumors developed in mice injected with uncloned P815-GPI-IL-12 cells, still express transfected-GPI-IL-12 in the absence of selection pressure, tumors were excised from one of the mice challenged with P815-GPI-IL-12 cells. Tumor cells were isolated by collagenase and dispase treatment and the cell surface expression of IL-12 and other antigens were determined by flow cytometry. The expression levels of MHC class I and CD54 were not altered, however, the expression of GPI-IL-12 was completely lost in these tumor cells (data not shown).

To determine whether the antitumor immune response induced by GPI-IL-12 was due to either systemic or local effect, mice were inoculated with live P815-GPI-IL-12 or P815-sec-IL-12 or wild type P815 cells or RPM medium alone. Serum samples were collected for 3 days and serum IL-12 and IFN-γ levels were estimated by sandwich ELISA. There was no difference in serum IL-12 levels between mice injected with P815-GPI-IL-12 or P815 cells or RPMI medium. However, under identical conditions, the serum IL-12 levels were increased about two fold after 3 days in mice injected with P815-secIL-12 cells (data not shown). It has been determined the serum IFN-γ as a measure of IL-12 in systemic circulation in these mice. Serum IFN-γ levels were the same in mice injected with P815-GPI-IL-12 or P815 cells or RPMI medium alone (data not shown). However, a time dependent increase in IFN-γ (2 and 4 fold at day 2 and 3 post-inoculation, respectively) was seen in mice injected with P815-secIL-12 cells. These findings suggest that the antitumor immune response induced by GPI-IL-12 may be mediated by local effect, whereas sec-IL-12 may act through entering the systemic circulation.

### Example 8

### Functional Incorporation GPI-Anchored Human IL-12 (GPI-ML-12) onto Human Tumor Cell Membranes

### Materials and Methods

Cell Lines, Monoclonal Antibodies and Cytokines.

Chinese hamster ovary cell line (CHOK1), mouse hybridoma against hCD3 (OKT3), hMHC class I (W6/32), rat hybridomas against hIL-12 (20C2), and a mouse myeloma cell line secreting X63), were purchased from ATCC (Manassas, Va.). Murine anti-human CD16 (CLBFcgran-1) and anti-human B7-1 (PSRM3) hybridoma cell lines were described earlier (Nagarajan, S., et al., J. Biol. Chem. 270:25762-25770 (1995); McHugh, R. S., et al., Clin. Immunol. Immunopathol. 87:50-59 (1998)). The following human tumor cell lines: melanoma (SKMEL28), Burkitt-lymphoma (RAJI, and JY), mammary carcinoma (MCF-7) and erythroleukemia (K562) were also purchased from ATCC. Human renal cell carcinoma cell line (RCC-1) and RCC-1 transfected with B7-1 (RCC-1.CD80) were established in our laboratory and described earlier (Wang, Y.-C., et al., J Immunother. 19:1-8 (1996)). RCC-1, SKMEL28 and MCF-7 cells were cultured in DMEM:F12 (1:1) supplemented with 5% FBS, 2 mM glutamax I (Invitrogen, Carlsbad, Calif.), 1 mM sodium pyruvate, penicillin (100 units/ml), and streptomycin (100 μg/ml), 55 μM β-mercaptoethanol, and gentamicin 50 μg/ml (cDF12). RCC-1.CD80 was maintained in cDF12 medium supplemented with G418 (400 μg/ml). The hybridomas and other cell lines were maintained in RPMI 1640 supplemented with 10% FBS (1-cyclone, Logan, Utah), 2 mM glutamine, and other additives at concentration mentioned above (cRPMI). All cell culture reagents were purchased from Mediatech Inc (Hemdon, Va.), unless indicated. Unconjugated and HRP- or FITC-conjugated-F(ab′)₂ goat anti-mouse IgG and F(ab′)₂ goat-anti-rat IgG were purchased Jackson Immunochemicals (West Grove, Pa.). Mouse anti-human IFN-γ mAbs (Clones 2G1 and B1313.5) were purchased from Pierce Endogen (Rockford, Ill.). Human IL-2 was from NCI cancer program, and human hIL-12, and IFN-γ were purchased from BD Pharmingen (San Diego, Calif.).

Construction of GPI-hIL 12 cDNA.

A mammalian expression vector cassette tCD80-CD59-pcDNA3^{neo} with GPI-anchor signal sequence of CD59 (Nagarajan, S., and Selvaraj, P., Cancer Res 62:2869-2874 (2002)) was used to construct cDNAs encoding the GPI-anchored form of human hIL-12 (GPI-hIL-12). hIL-12 is a disulfide-linked heterodimer consisting of 35 and 40 kDa polypeptides. The coding regions of p35 and p40 (excluding the stop codon) subunits of hIL-12 were PCR amplified using pNKSF-35, pNKSF40 (ATCC) as templates using Pfu DNA polymerase (Stratagene, La Jolla, Calif.). The primers to amplify p35 cDNA were, forward (catccagcagctcctctca) and reverse (cattgcttaaggagctcagatagccc); and the following forward (gcacatcagaccaggcagct) and reverse (ccattgcttaaggatcggaccctgcagggaa) primers were used to amplify cDNA encoding p40 kDa subunit of hIL-12. The reverse primers were designed to have an Afl II linker (underlined). The tCD80 cDNA was excised with EcoR V/Afl II leaving the GPI-anchor addition signal sequence of CD59 with the vector cassette. The Afl II-digested PCR products of p35 and p40 kDa cDNAs were then cloned into the cassette containing GPI-anchor addition signal sequence of CD59 at EcoR V/Afl II sites. Using this strategy, both p35-CD59 and p40-CD59 cDNAs were cloned into pcDNA3^{neo} mammalian expression vector, and the p35-CD59 cDNA was further subcloned into pUB6^{blasticidin} (pUB6^{bla}) vector (Invitrogen, Carlsbad, Calif.).

Establishing Transfectants Expressing GPI-Anchored hIL-12.

CHOK1 transfectants expressing GPI-hIL-12 (CHO-GPI-hIL-12) was established by co-transfecting p35-CD59-pUB6^{bla} (1 μg) and p40-CD59-pcDNA3^{neo} (1 μg) codas using Fugene (Roche Biochemicals, 1N), according to the manufacturer's instruction. K562 transfectants expressing GPI-hIL-12 were established by co-transfecting human p35-CD59-pUB6^{bla} (10 μg) and p40-CD59-pcDNA3^{neo} (10 μg) cDNAs by electroporation using a BioRad gene pulser II (Hercules, Calif.). The electroporation was performed using the cells in serum free RPMI 1640 pulsed at 960 μF and 0.25 kV/cm. After 48 h of transfection, the GPI-hIL-12⁺ cells were enriched by biomagnetic selection using anti-hIL-12 mAb (20C2) and sheep anti-rat IgG magnetic beads (10 beads/cell) and two cycles of panning method as described earlier (McHugh, R. S., et al., Proc. Natl. Acad. Sci. USA 92:8059-8063 (1995)). The enriched GPI-hIL-12+ cells were cultured in complete cRPMI containing blasticidin (10 μg/ml) and G418 (800 μg/ml). K562 cells were further subcloned. To determine the cell surface expression of hIL-12, cells were stained with anti-hIL-12 mAb, and analyzed using a FACScan flow cytometer (Becton-Dickinson, San Jose, Calif.). The GPI-linkage of hIL-12 was confirmed by treating CHO-GPI-hIL-12 cells with phosphatidylinositol-specific phospholipase C(PIPLC) (Nagarajan, S., and Selvaraj, P., 2002, supra) followed by flow cytometric analysis.

Establishing CHO cells expressing costimulatory molecules. CHO cells expressing human GPI-CD80 were described earlier (Poloso, N., et al., Vaccine 19:2029-2038 (2001)). CHO cells expressing GPI-CD40 was established by transfecting CD40-CD59 cDNA. GPI-anchored CD40 was constructed using the CD59 cassette, as described above. Briefly, total RNA was isolated from Raji cells and reverse transcribed using oligodT and Superscript RT II (Invitrogen). cDNA encoding the extracellular domain of CD40 was PCR amplified from 2 μl of the reverse transcribed mix and Pfx DNA polymerase (Invitrogen). The following forward (5′-tataaagctttcacctcgccatggtt) and reverse (5′ attgcttaagctcagccgatcctgggga) primers were used to amplify the extracelluar domain of CD40. A HindIII and AJlII restriction sites (underlined) were introduced in the forward and reverse preimers, respectively. The resultant chimeric construct (hCD40-CD59) was expressed in CHOK1 cells. Cell surface expression was analyzed by flow cytometry after staining the cells with anti-hCD40 mAb (FGK45) and FITC conjugated-goat anti-mouse IgG.

Western blot analysis. CHOK1-GPI-hIL-12 transfectants were washed in PBS and cell surface proteins were biotinylated using sulfo-NHS biotin for 30 min at 4° C. as described earlier (Lisanti, M. P., and Sargiacomo, M. 1998. Biotinylation and analysis of membrane-bound and soluble proteins. Current Protocols in Immunology 2:8.16.11-18.16.15 (1998)). Cells were washed and lysed in 50 mM Tris-HCl pH 8.0 containing 1% octyl β-glucoside, 5 mM iodoacetamide, 1 mM PMSF and 1% aprotinin for 1 h at 4° C. Lysates were precleared using sheep anti-mouse conjugated magnetic beads M280 (Dynal, Lake Success, N.Y.). GPI-hIL-12 was immunoprecipitated using anti-human hIL-12 mAb (clone name), coupled to sheep anti-mouse conjugated magnetic beads M280. GPI-hIL-12 was eluted in 50 mM Tris-HCl, pH 6.8/0.5% SDS and subjected to SDS-PAGE under non-reducing conditions. Proteins were electrotransferred to Biotrans PVDF membrane (ICN, Costa Mesa, Calif.). Blot was developed using biotinylated-goat-anti-human hIL-12 polyclonal antibody followed by streptavidin-HRP and proteins were visualized by chemiluminescence. Recombinant soluble hIL-12 was used as a positive control in western blot analysis.

Proliferation Assays.

Proliferation of PHA-activated human T cells. Peripheral blood mononuclear cells (PBMC) were isolated from normal healthy donor as described (McHugh, R. S., et al., Proc. Natl. Acad. Sci. USA 92:8059-8063 (1995); Wang, Y.-C., et al., J. Immunother. 19:1-8 (1996)). T cells were enriched from PBMC by plate adherent and negative selection after staining the cells with anti-CD32, anti-CD16 and anti-CD19 mAb followed by using goat anti-mIgG magnetic beads (Polysciences, PA). PHA-activated human T cells were prepared using 1% PHA (Invitrogen, Carlsbad, Calif.) by standard procedure (Gately, M. K., et al., Curr Protocols Immunol 1:6.16.11-16.16.15 (1995)). CHOK1 and CHO-GPI-hIL-12 cells (stimulators) were treated with mitomycin C (50 μg/ml) for 30 min at 37° C., washed extensively with complete RPMI and used in the proliferation assays. PHA-activated T cells (responders) were co-cultured with mitomycin C-treated stimulator cells for 48 h. Cells were pulsed with 3H-thymidine (1 μCi/well) (Amersham, Arlington Heights, Ill.) for the final 18 h and harvested using a Packard filtermate cell harvester (Meriden, Conn.). ³H-Thymidine uptake was counted in a Packard Top count microplate scintillation and luminescence counter (Downers Grove, Ill.).

Mixed lymphocyte reaction assay. An allogeneic mixed lymphocyte reaction (MLR) assay was carried out to determine the efficacy of GPI-hIL-12 to induce alloantigen specific T cell stimulation. Mitomycin C-treated PBMC (1×10⁵) from one normal donor was co-cultured with unactivated T lymphocytes (1×10⁵) from another donor for 72 h. Mitomycin C-treated CHOK1 or CHO-GPI-hIL-12 cells (1×10³) were added and further incubated for 48 h. Cells were pulsed with ³H-thymidine (1 μCi/well) for the final 18 h and uptake of ³H-thymidine was determined as described above. Recombinant soluble hIL-12 (rechIL-12) was included as a positive control.

IFN-γ release assay. PHA-activated T cells were co-cultured with mitomycin C-treated CHO-GPI-hIL-12 cells for 48 h as described under proliferation assay. Cultures were centrifuged and supernatant was analyzed for the release of IFN-γ to determine the hIL-12-dependent T cell stimulation. IFN-γ release was determined by sandwich ELISA using corresponding mAb pairs. CHO cells or rechIL-12 were used as negative and positive controls, respectively.

Preparation of Isolated Membrane Vesicles from CHO-GPI-hIL-12 Transfectants.

Isolated membrane vesicles were prepared from CHOK1 and CHO-GPI-hIL-12 cells by sucrose gradient ultracentrifugation (Poloso, N., et al., Vaccine 19:2029-2038 (2001)). Briefly, cell pellets were homogenized on ice in cold solubilization buffer (20 mM Tris pH 8.0 containing 10 mM NaCl, 0.1 mM MgCl₂, 0.02% NaN₃ and 0.1 mM PMSF) and ultracentrifuged (93,000×g) for 1 hour over a 41% sucrose gradient. The interface was recovered and washed three times in solubilization buffer by centrifugation. Membranes were resuspended in protein free RPMI with antibiotics and frozen in aliquot at −80° C. Protein concentrations of membranes were determined by BioRad dye binding method using BSA as standard. Expression of GPI-hIL-12 on the isolated membranes was determined by ELISA using anti-hIL-12 mAb. Color was developed using TMB-1 as substrate, and reaction was stopped with 2NH₂SO₄. The color developed was read at 415 nm in an ELISA microplate reader (Molecular Devices, Sunnyvale, Calif.). Isolated membranes prepared from CHOK1 cells were used as a negative control. Analysis of Functional integrity of isolated GPI-hIL-12 positive membranes. The functional integrity of isolated GPI-hIL-12 positive membranes was analyzed in a T cell proliferation assay using PHA-activated T cells. Membranes prepared from CHO GPI-hIL-12 and CHOK1 cells were used in the proliferation assay. Augmentation of T cell proliferation in a MLR assay was also performed to determine the functional integrity of the isolated GPI-hIL-12 positive membranes. In all the assay different concentration of membranes were used. Recombinant soluble hIL-12 was used as positive control.

Purification of GPI-hIL-12. Large-scale purification of GPI-anchored-hIL-12 was performed from cell lysates of K562-GPI-hIL-12 cells. Briefly, K562-GPI-hIL-12 cell pellets (3 g) were purified by immunoaffinity chromatography using anti-hIL-12 mAb (20C2)-NHS-activated agarose column. Briefly, K562-GPI-hIL-12 cell pellets were lysed in 10 volumes of 50 mM Tris-HCl pH 8.0 containing 0.3% saponin, with a cocktail of protease inhibitors and 5 mM 1,10, phenanthroline for 30 min at 4° C. Equal volume of lysis buffer containing 50 mM Tris-HCl pH 8.0 containing 2% Triton X-100, with a cocktail of protease inhibitors and 5 mM 1,10-Phenonthroline was then added and further incubated for 30 min at 4° C. After clearing the cell debris at 2000×g for 10 min, the supernatant was centrifuged at 100,000×g for 1 h and the supernatant was passed through 20C2-Sepharose, anti-hIL-12 mAb column. The column washed with 50 volumes of 50 mM Tris-HCl pH 8.0/200 mM NaCl/1% Triton X-100, and eluted in 100 mM glycine-HCl pH 3.0/150 mM NaCl/0.1% octyl s-glucoside. Fractions were then tested for the purified GPI-hIL-12 by ELISA. Active fractions were pooled and dialyzed against HBSS/0.01% octyl 3-glucoside prior to protein transfer onto tumor cells.

Protein Transfer of Purified GPI-hIL-12 Onto Tumor Cells.

Isolation of tumor cell membranes from human tumor cells was carried out as described earlier (Poloso, N., et al., Vaccine 19:2029-2038 (2001)). Protein transfer of purified GPI-hIL-12 onto tumor cells or isolated tumor cell membranes were done as described earlier for GPI-B7 (Id.). Briefly, protein transfer of GPI-hIL-12 was done using isolated tumor cell membranes (20 μg protein) in HBSS (Ca and Mg free)/0.1% ovalbumin were incubated with either elution buffer (as control) or purified GPI-hIL-12 for 4 h at 37° C. Cells and isolated cell membranes were then washed in HBSS/5 mM EDTA. Cell surface expression of GPI-hIL-12 was then determined by flow cytometry whereas membrane incorporation was determined by ELISA as described earlier, using anti-hIL-12 mAb. As a control, MHC class I expression on the membranes were also determined using a non-polymorphic anti-hMHC class I mAb (W6/32). HRP-conjugated goat anti-rat IgG or goat anti-mouse IgG were used as the detecting antibody for human hIL-12 and MHC class I, respectively. Color was developed using 3; 3; 4,4; -tetramethyl benzidine (100 μl/well) substrate. The reaction was stopped by the addition of equal volume of 2N H₂SO₄ and the color developed was read at 450 nm in a Microplate reader (Molecular Devices, CA). Similarly, protein transfer of GPI-hIL-12 onto human tumor cells was done. Human tumor cells (2×10⁶) were mitomycin C treated for 30 min. After extensive washing mitomycin-C treated cells in HBSS (Ca and Mg free)/0.1% ovalbumin were incubated with elution buffer (negative control) or purified GPI-hIL-12 for 2 h at 37° C. Cells were then washed in HBSS/5 mM EDTA and used for functional assay.

Analysis of Functional integrity of protein transfer modified-GPI-hIL-12 positive tumor cell membranes. The functional integrity of isolated GPI-hIL-12 positive membranes was analyzed in a T cell proliferation assay by measuring the proliferation of activated-T cells and IFN-γ release by the activated-T cells. Human tumor cells or isolated tumor cell membranes expressing GPI-hIL-12 were co-cultured with membranes prepared from CHO GPI-hIL-12 and CHOK1 cells were used in the proliferation assay as described above. Augmentation of T cell proliferation in a MLR assay was also performed to determine the functional integrity of the isolated GPI-hIL-12 positive membranes. In all the assay different concentration of membranes were used. Recombinant soluble hIL-12 was used as positive control.

### Results

This example shows that chimeric hIL-12-CD59 cDNA transfected cells express GPI-anchored IL-12 heterodimeric protein on the cell surface. The cDNAs encoding the entire coding region of p35 and p40 subunits of human hIL-12 were ligated in-frame to a GPI-anchor addition signal sequence of CD59 in a mammalian expression vector cassette. CHOK1 stable transfectants expressing GPI-hIL-12 were established by co-transfecting chimeric cDNAs of p35 and p40 subunits, as described under methods. Flow cytometric analysis of the CHO-GPI-hIL-12 transfectants showed cell surface expression of hIL-12. More than 90% of the GPI-hIL-12 protein expressed on transfected cells was released by PIPLC treatment, indicating that the hIL-12 is anchored to the cell surface via a GPI-moiety.

The heterodimeric nature of the chimeric GPI-hIL-12 protein was determined by immunoprecipitation of GPI-hIL-12 followed by SDS-PAGE analysis. Western blot analysis of both GPI-hIL-12 showed a protein band corresponding to 80 kDa under non-reducing conditions. Under reducing conditions, two bands corresponding to 35 and 40 kDa were seen. The relative mobility of GPI hIL-12 expressed on CHOK1 cells was higher than the standard recombinant hIL-12. This difference in the mobility could be due to difference in the additional molecular weight from GPI-moiety and cell specific glycosylation. These results indicate that the GPI-hIL-12 was processed and folded correctly and was expressed as a disulfide-linked heterodimer.

This example also demonstrates that cell surface expressed GPI-hIL-12 induces T-cell proliferation. The functional integrity of GPI-hIL-12 to induce T cell proliferation was determined by co-culturing activated-T cells with CHO-GPI-hIL-12 cells. IL-12 has been shown to induce the proliferation of activated-T cells (Schoenhaut, D. S., et al., J Immunol 148:3433-3440 (1992)). CHO-GPI-hIL-12 cells induced T-cell proliferation and the proliferation was dependent on the number of stimulator cells used in the assay. The level of proliferation induced by GPI-hIL-12 was identical to the level that obtained using 0.5 ng/ml of recombinant soluble hIL-12. To determine that the proliferation is induced by the GPI-hIL-12 heterodimeric on the cell surface, CHO cells expressing either GPI-anchored p35 or GPI-anchored p40 kDa proteins were also established. Under the similar conditions, CHO cells expressing either one of the subunits of hIL-12 as GPI-linked did not induce T cell proliferation. This proliferation was completely blocked by the blocking antibody against hIL-12. Moreover, under the similar conditions wild type CHOK1 or CHOK1 transfected with GPI-mICAM-1 did not induce proliferation of PHA-activated T cells. These results indicate that the chimeric GPI-hIL-12 is functionally active and can deliver the signal necessary for the induction of T-cell proliferation.

hIL-12 act only on activated T cells and as an adjuvant to enhance the proliferation of activated-T cells. Moreover, it has been shown that IL-12 can augment T cell proliferation induced by costimulatory molecules such as CD80 (Pulaski, B. A., et al., Cancer Immunol Immunother 49:34-45 (2000); Chen, P. W., et al., Ann. NY. Acad. Sci. 795:325-327 (1996); Coughlin, C. M., et al., Cancer Res. 55:4980-4987 (1995)). Therefore, the ability of GPI-hIL-12 to enhance the T cell proliferation that was primarily initiated by costimulatory molecules was determined. An isolated system of CHOK1 cells transfected with CD80, CD40 or CD80/CD40 double transfectants were used in this study. CD80 alone can induce the proliferation of activated T cells, whereas CD40 did not induce any proliferation. Similarly, transfectants expressing CD40/CD80 showed about a 2 fold increase in proliferation of activated T cells. When CHO-GPI-hIL-12 was included in this assay, it increases the proliferation further to about 2.5 fold. This finding indicates that GPI-hIL-12 can enhance or augments the proliferation that was initiated by costimulatory molecules such as CD80 and CD40.

This example further shows that membrane vesicles isolated from CHO cells expressing GPI-hIL-12 induce proliferation of activated T cells. The isolated tumor cell membranes can therefore be modified to express GPI-hIL-12 administered at the vaccine injection site for efficient local delivery. Moreover, isolated tumor membranes can be modified to express hIL-12 by protein transfer approach using GPI-hIL-12. Earlier report have shown that isolated tumor cell membranes can be prepared from tumor cell or surgically removed tumor tissue (Poloso, N., et al., Vaccine 19:2029-2038 (2001)). These membranes could be modified to express GPI-anchored B7-1 and ICAM-1 molecules by protein transfer and the expression of GPI-anchored proteins have shown to be stable (Id.). Prior to using the purified GPI-hIL-12 in protein transfer experiments, we prepared the isolated cell membranes from CHO-GPI-hIL-12 and determined whether it can induce proliferation of T cells in a PHA-activated T cell and allogenic T cell proliferation assays as described under methods. Isolated cell membranes expressing GPI-hIL-12 enhanced proliferation of PHA activated cells in a dose dependent manner. Under similar conditions, cell membranes prepared from untransfected CHO cells did not have any effect. To compare the level of T cell proliferation induced by GPI-hIL-12 membranes, soluble recombinant hIL-12 was included in the proliferation assay. The level of proliferation was linear up to 0.5 pg/ml. The proliferation induced by the GPI-hIL-12 positive isolated cell membranes (at 40 μg/ml membrane protein) is equivalent to that seen with 0.5 pg/ml soluble hIL-12.

This example further shows GPI-hIL-12 expressed on isolated cell membranes is capable of augmentation of allogenic T cell proliferation. The induction of allogenic T cell proliferation by isolated cell membranes expressing GPI-hIL-12 was determined. hIL-12 has been shown to act as an adjuvant to enhance the induction of T cell proliferation (Scott, P., and Trinchieri, G., Semin Immunol 9:285-291 (1997)). A MLR assay was performed by mixing T cells with mitomycin C treated allogenic PBMC. GPI-hIL-12 membranes were added 3 days after the initiation of the MLR assay. Membranes from CHO cells and soluble recombinant hIL-12 were used as negative and positive controls, respectively. Addition of GPI-hIL-12 isolated cell membranes showed about 30 fold increases in allogenic T cell proliferation over the CHO cell control. The level of enhancement is similar to that seen with 0.5 pg/ml of soluble recombinant hIL-12. These findings indicate that the isolated cell membranes expressing GPI-hIL-12 is functional in inducing T cell proliferation.

This example further demonstrates that purified GPI-hIL-12 can be incorporated onto isolated tumor cell membranes by protein transfer method. As mentioned earlier, another potential application of GPI-anchored cytokines is to modify tumor cells or isolated tumor cell membranes with purified GPI-anchored cytokines. Therefore, to determine whether GPI-IL-12 could be used to modify tumor cell membranes by protein transfer, GPI-hIL-12 was purified from K562-GPI-hIL-12 cell lysates by a single step immunoaffinity chromatography, as described under methods. SDS-PAGE analysis of the eluted proteins showed a major band of about 80 kDa under non-reducing conditions and low levels of contaminating proteins. Under reducing conditions, two bands corresponding to 35 and 40 kDa was seen, indicating the GPI-hIL-12 is expressed as a heterodimeric protein. Modification of isolated tumor cell membranes with purified GPI-IL-12 by protein transfer method was then determined. Isolated tumor cell membranes were prepared from various human tumor cell lines and incubated with purified GPI-hIL-12 as described under methods. The expression of GPI-hIL-12 on the isolated membranes was determined by ELISA using specific mAbs against hIL-12. All the tumor membranes could be modified to express GPI-hIL-12 by protein transfer. The levels of expression between different cells varied. This could be due to the variation in the lipid profiles on the biomembranes of different cells. These findings suggest GPI-hIL-12-has the intact GPI-anchored tail and can be used to modify tumor cell membranes by protein transfer.

The functional integrity of the protein transfer modified membrane in the T cell proliferation assay was also tested. The GPI-hIL-12-modified membranes induced proliferation of activated T cells. In addition IL-12 is known to induce the release of IFN-γ, therefore, the release of IFN-γ by the activated T cells using GPI-hIL-12 modified tumor cell membranes were then tested. GPI-hIL-12 modified membranes induced release of IFN-γ by activated T cells.

### Example 9

### Breast Cancer Vaccination

This study involved the transfection of murine mammary cells and the establishment of stable transfectants expressing IL-2, B7.1, and/or IL-12. Once stable expression of the immunostimulatory molecules was established, the antitumor effects of the membrane-bound molecules were investigated in which mice were directly challenged with wild-type or transfected tumor cells. The study phases are described in detail below.

In phase I, murine mammary tumor cells were transfected to express B7, IL-2, and IL-12 alone or in combination (upper panel). In Phase II, mice were challenged (n=5/group) with transfected or modified tumor cells and monitored for tumor growth (middle panel). In Phase III, tumor-free mice were rechallenged with wild-type cells and monitored for tumor growth (lower panel).

I. Establishment of tumor cell lines expressing immunostimulatory molecules: 4T07 tumor cell lines expressing IL-2, IL-12 and B7.1 immunostimulatory molecule combinations were established via transfection of cDNA and selection of high protein expressing cells via magnetic activated cell sorting (MACS), the panning method, and fluorescent activated cell sorting (FACS). FACS flow cytometry analysis was used to verify protein expression, and the enzyme phosphatidylinsotisol phospholipase-C(PIPLC) was used to confirm the GPI-linkage of the cytokine molecules. Finally, growth rate analysis was performed to verify that the transfected cell lines still grew at the same rate as the wild-type, parental tumor cells.

II. Direct challenge of mice with wild-type and transfected tumor cells: Mice were directly challenged with wild-type or transfected tumor cells to investigate the tumor growth properties and possible antitumor immune induction.

III. Secondary challenge and memory study: Mice that were tumor-free thirty days after direct challenge with the transfected tumor cells were subsequently rechallenged with wild-type 4TO7 cells to determine possible induction of antitumor immune memory.

### Materials and Methods

Cell Culture.

The murine mammary tumor line 4T07 was maintained in DMEM-F12 supplemented with 10% fetal bovine serum FBS), 2 mM L-glutamine, 1 mM sodium pyruvate, gentamicin (Sigma-Aldrich, 10 mg/ml solution with 2.5 ml/500 ml media), and penicillin/streptomycin (Invitrogen; 10 mg/ml stock solution with 2 ml/500 ml media). Media and reagents were purchased from MediaTech, Inc. (Herndon, Va.) unless indicated. Transfected 4T07 cell lines were maintained in culture media plus the blasticidan selection agent (Invitrogen; 10 μg/ml solution of blasticidan; 1 μL bla/1 ml media). Cells were incubated in a CO₂ incubator.

cDNA and Vectors.

cDNA encoding murine IL-2, IL-12 and B7 were previously constructed as described in Nagarajan S, Selvaraj P. Cancer Res 2002; 62:2869-2874. IL-2 and EL-12 cDNA were linked with a GPI-anchor signal sequence, as previously described by Nagarajan (Nagarajan S. Selvaraj P. Cancer Res 2002; 62:2869-2874) and McHugh (McHugh R S, et al., Proc Natl Acad Sci USA 1995; 92:8059-8063). The cDNA was ligated into individual puB6 vectors.

Establishment of 4T07 Transfectants.

The 4T07 mammary tumor cell line was transfected using FuGene6 transfection reagent (Roche Molecular Biochemicals) and selected with blasticidan (10 μg/ml). 1 μg of puB6 vector containing immunostimulatory molecule cDNA was transfected into the 4T07 cells. Single transfectants received 1 μg total of cDNA, while double transfectants received 1 μg of each cDNA vector. 4T07 cells were transfected to express B7, GPI-IL-2, GPI-IL-12, GPI-IL-2 and GPI-IL-12, B7 and GPI-IL-2, and B7 and GPI-IL-12. To augment population of cells expressing the costimulatory molecules, the cells were sorted by antibody-conjugated magnetic beads and were panned against anti-immunostimulatory molecule antibodies. Surface expression of IL-2, B7 and IL-12 was analyzed using fluorescent activated cell sorting (FACS) and the FACScan flow cytometer.

Magnetic Activated Cell Sorting (MACS) Selection.

MACS selection is a physical selection method for cells that utilizes antibodies conjugated to magnetic beads. Briefly, cells are incubated with protein-specific primary antibodies and subsequently incubated with antibodies specific to the primary antibody. These secondary antibodies are conjugated to tiny magnetic beads. A magnet is then used to select the magnetic bead-bound cells of interest.

Non-stringent selection of protein expression in 4T07 transfectants was performed by coincubation of cells with anti-mouse(m) antibodies (S4B6, rat anti-mIL-2; IG10, rat anti-mB7; C17.8, rat anti-mIL12) and subsequent coincubation of cells with Sheep anti-Rat IgG magnetic beads (Dynal Biotech Dynabeads). Cells were dissociated from flasks using 0.05% trypsin/EDTA (MediaTech, Inc.) or PBS/5 mM EDTA, spun at 1200 rpm for 5 minutes, and counted using a hemocytometer. 1-2×10⁶ cells were resuspended at 200 μl sterile DMEM/5 mM EDTA/1×10⁶ cells and added to a sterile 1.5 ml tube. 200 μl of primary antibody culture supernatant was added to the cell suspension and incubated, with shaking, at 4° C. for 30 minutes. Cells were then spun at 1200 rpm for 5-7 minutes and washed 2× with DMEMIEDTA. Cells were then resuspended in 150 μl DMEM/EDTA/1×10⁶ cells and 25 μl beads/1×10⁶ cells to yield a ratio of 10 beads per cell. The cell suspension was then incubated with shaking at 4° C. for 30 minutes. The tubes were then placed against a MACS separation unit magnet (Miltenyi Biotech), and the supernatant was aspirated. The tube was removed from the magnet, 1 ml culture media added, and cells resuspended. The magnetic bead separation was carried out for a total of three times, and upon the final resuspension, cells were cultured into a T25 flask in 10 ml culture media with selection agents.

Panning Technique.

The panning technique utilizes antibodies bound to the surface of a bacterial petri dish to select for cells with high protein expression. Primary antibodies adhere to secondary antibodies bound to the plastic bottom of the petri dish. Cells are added to the dish, and protein-expressing cells adhere to the primary antibodies. All other cells are washed away.

Cells were panned against one antibody as a time, and all reagents were sterile filtered and handled inside the tissue culture hood. Bacterial petri dishes (Falcon 1029) were coated with 5 ml of 10 μg/ml (first panning) or 2 μg/ml (second panning) of rabbit anti-rat IgG antibodies (diluted in sterile, cold PBS). The plates were left to sit on a flat surface overnight at 4° C. (cold room), after which the antibody solution was removed and stored for subsequent panning. 10 ml ice cold PBS was added to the plates for 2-3 minutes to remove any nonbound IgG. 4-5 ml of rat anti-mouse antibody (S4B6, anti-IL-2; IG10, anti-B7; C17.8, anti-IL-12) culture supernatant was added to the plates and incubated at room temperature for 30 minutes. Panning for the cells expressing two immunostimulatory molecules had to be done twice, once per immunostimulatory molecule.

During the primary antibody incubation, transfected 4T07 cells in T75 culture flask were dissociated using 5 ml PBS/EDTA or 0.05% trypsin/EDTA, centrifuged, resuspended in 5 ml ice cold culture media and kept on ice. 1 ml of cell suspension was recultured as backup, and 1 ml of media replaced. 50 μl of 500 mM ice cold EDTA was added to cell suspension. Inside culture hood, the antibody solution was removed and stored, and the cells were transferred to plates and placed at 4° C. for 45 minutes (no shaking). After 15 minutes, the plates were rotated 180° to ensure equal distribution of cells and left for the remaining 30 minutes. After the incubation, inside the culture hood, the media was removed and 10 ml ice cold DMEM/EDTA was added gently (same-spot marked with an X). The washing was repeated for a total of 3 times. The plates were checked under microscopy for attached cells. 3 ml of culture media was added, and cells were detached using a transfer pipette and transferred to a T25 culture flask. Cell detachment was repeated for a total of 3 rinses. The cells were cultured in selection media and, after the cell density increased, and analyzed via flow cytometry for protein expression.

Flow Cytometry Analysis.

The fluorescent activated cell sorting (FACS) staining technique utilizes fluorescently labeled antibodies to detect surface protein expression. Briefly, cells are stained one of two ways: with a primary antibody and a conjugated secondary antibody, or with a conjugated primary antibody. In the former case, cells are incubated with an antibody specific to the surface protein of interest and then subsequently incubated with a secondary antibody specific to the primary antibody used. The secondary antibody is conjugated to a fluorescent protein, such as PE (R-phycoerythrein) or FITC (fluorescein isothiocyanate), which will fluoresce when excited with a beam of light of a specific wavelength or frequency. In the latter method, the primary antibody is already conjugated with the fluorescent protein, and only one antibody/cell incubation is necessary. It is possible to stain for two proteins at once if the primary antibodies for each are conjugated for proteins that fluoresce different colors, that is, at different frequencies (e.g., PE is red and FITC is green). It is also possible to stain for internal proteins if the cell membranes are first permeabilized. The flow cytometry machine (Becken Dickenson) takes the stained cell population and passes the cells through a small opening in a small stream the width of one cell. The machine shines a beam of light on the stream of cells and the computer registers the fluorescent intensity of the signal. When compared with the background fluorescence of an unstained control cell population, the measured fluorescence can indicate both the size of the protein-positive population (given as percent positive) as well as the relative protein concentration (given as the total mean peak fluorescence intensity). All values reported in this study for percentage positive and mean peak fluorescence intensity have subtracted the background fluorescence in the corresponding negative control population (i.e, unstained control cells).

Reagents: Fluorescent Activated Cell Sorting (FACS) buffer (PBS/5 mM EDTA/1% FCS/0.02% Azide), PBS/EDTA or 0.05% trypsin/EDTA, 2% formalin in PBS (10 ml of 37% formaldehyde with 175 ml PBS, stored in brown bottle at room temperature), primary antibodies (rat anti-mouse IgG's: S4B6, IG10, C17.8) and secondary antibodies (FITC-conjugated goat anti-rat IgG). Directly conjugated anti-mouse antibodies were also used: rat anti-mouse IL-2-PE, IL-12-PE, IL-12-APC, and B7-FITC (BD Biosciences). 250 μl FACS buffer was added to each well used in a 96 well v-bottom microtiter plate or 1 mL eppendorf tubes and incubated for 10 minutes (minimum) at room temperature. The cells were dissociated from flask with PBS/EDTA or 0.05 trypsin/EDTA, centrifuged, and resuspended in 5-6 ml culture media. 1 ml of cell suspension was recultured, and the rest of the cells were centrifuged again and resuspended in 5 ml FACS buffer. The cell count was taken on a hemocytometer (20 μl cell suspension and 20 μl trypan blue dye), and cell viability had to be 90% to continue. (Cell count×10⁴× dilution factor of 2=#cells/ml). Cells were resuspended in ice-cold FACS buffer to give 5×10⁶ cells/ml.

The following steps were done on ice. When using conjugated secondary antibodies, 50 μl of the cell suspension were added per well, and 50 μl of primary mouse antibody added to cells. The 96 well plate was sealed with a plate cover and shaken at 4° C. for at least 30 minutes. The plate was centrifuged for 1 minute at 1500 rpm (Jouan centrifuge). To remove the unbound antibodies, a multiwell aspirator was used to remove the supernatant, 200 μl FACS buffer was added to cell pellet, and the pellet was resuspended with multichannel pipette. The plate was centrifuged again and the wash repeated. 50 μl of 1:50 diluted (in FACS buffer) FITC-conjugated secondary antibody was added to cell pellet and the pellet resuspended. The plate was covered and shaken at 4° C. for 20-30 minutes. Following incubation, the cells were washed 1-2 times with 200 μl FACS buffer. The pellet was resuspended in 150 μl cold FACS buffer, followed by the addition of 150 μl 2% formalin, and the solution was mixed immediately. The samples were removed from microtiter plates and placed into labeled microtubes in microtube box covered with aluminum foil. The tubes were stored in refrigerator for a maximum of 1-2 days until the data was acquired using FACScan machine (Becken Dickenson) and the CellQuest analysis software.

In FACS analyses performed after the transfected tumor cell lines were established, directly conjugated primary anti-mouse antibodies were used. 2.5-5×10⁵ cells/100 μl were washed once with 150 μl FACS buffer and then stained with a 1:50 dilution of the antibody in FACS buffer. The cells were incubated for at least 20 minutes in 4° Celcius and washed 1-2× with 150 μl FACS buffer. The pellets were ultimately resuspended in 150 μl cold FACS buffer, followed by the addition of 150 μl 2% Formalin/PBS with immediate mixing. FACScan analysis proceeded as described.

Fluorescence Activated Cell Sorting.

Briefly, cells are stained with fluorescently-labeled, protein-specific antibodies in a manner than ensures cell viability (e.g., cells are not fixed in formalin as in standard FACS staining). The cell sort equipment analyzes the intensity of the fluorescent signal in the cell population and directs the positive-staining cells from the cell stream into a collection tube instead of into the waste container. With the computer analysis, one is able to gate upon the cell population of interest (e.g., high-expressing cells), thus selecting a specific cell population and eliminating the rest.

Cells were dissociated with 0.05% trypsin/EDTA, centrifuged, and counted in culture media using a hemocytometer. The cells were then resuspended in cell sort wash buffer (culture media/5 mM EDTA) to give 1×10⁶ cells/ml. 5×10⁵ cells were set aside as an unstained control. Antibodies, as described in the FACS Flow Cytometry section, were added at a 1:50 dilution to the cell suspension. Cells were stained with the antibodies for 30 minutes with shaking at 4° C. Cells were washed once with wash buffer and resuspended in buffer at 1×10⁷ cells/ml. Cells were then taken to the cell sorting facility in the Emory University Hospital for sorting. Collection media for the sorted cells was DMEM/F12/50% FBS.

PIPLC (phosphatidylinositol phospholipase-C) treatment.

PIPLC is an enzyme that cleaves the lipid portion of a GPI-anchor and is used to verify GPI-linkage of proteins. Eppendorf tubes (1.5 ml) were pre-rinsed with 1 ml of PIPLC buffer (PBS/0.1% Ovalbumin (1 mg/ml)). The cells were dissociated with 0.05% trypsin/EDTA, counted with a hemocytometer to ensure >90% viability, and resuspended at 10×10⁶ cells/ml in cell buffer (2 parts PBS/EDTA: 1 part RPMI/10% FBS). 500 μl of PIPLC buffer was added to the prerinsed eppendorf tubes, and 100 μl of cell suspension (1×10⁶ cells) was added to each tube. Tubes were centrifuged for 10 seconds at 10,000 rpm in a microcentrifuge, and cells were resuspended in 50 μl of PIPLC Buffer. PIPLC enzyme (Glyko) was diluted 1:1000 in PIPLC buffer (1 μl in 1 ml), and 50 μl of diluted PIPLC was added to the cell suspension. The enzyme and control tubes (no PIPLC enzyme added) were incubated for 45 minutes in a 37° C. water bath with tapping of the tubes every 10 minutes to ensure mixing of the enzyme with the cells. At the end of the incubation, the cells were centrifuged and washed with the addition of 1 ml of FACS buffer (PBS/EDTA/1% FCS/0.02% Azide). The pellet was re-suspended in 100 μl of FACS buffer, and 1 ml FACS buffer was then added for another wash. From here, the cells underwent the FACS staining protocol to determine surface protein expression.

CFSE Staining to Determine Cell Growth Rate.

CFSE (5,6-carboxyfluorescein diacetate succinimidyl ester) is a small molecule that easily diffuses into cells and couples to cytoplasmic proteins (Chen J C, et al., J Immunol Methods 2003; 269:123-133). The molecules are membrane-permeant, and by tracking the fluorescence of a cell population, it is possible to examine its growth rate based upon the decrease in CFSE expression per cell. CFSE staining procedure was adapted from methods of Chen (Id.) and Lyons (Lyons, AB, Immunol and Cell Biol 1999; 77:509-515). CFSE (Sigma-Aldrich, 100 mg) was brought to a 100 mM stock concentration in DMSO and subsequently diluted to 1 mM DMSO, stored in −20° C., and protected from light. Cells were dissociated from flasks, counted with a hemocytometer, and resuspended at 1×10⁷ cells/ml in sterile PBS. 2-5×10⁵ cells were set aside, resuspended in 150 μl resuspended at 1×10⁷ cells/ml in sterile PBS. 2-5×10⁵ cells were set aside, resuspended in 150 μl FACS buffer and fixed with 150 μl 2% formalin. These cells served as the unstained control population for FACS analysis. The CFSE was diluted 1:100 to a 1001 mmol/L concentration directly into the volume in which the cells were resuspended. The cells were incubated with CFSE for 10 minutes at room temperature with occasional agitation to ensure complete mixing. The reaction was quenched with 1 ml culture media (containing FBS) and let sit for 1 minute at room temperature. Again, 2-5×10⁵ cells were set aside, washed 1× with FACS buffer, resuspended in 150 μl FACS buffer, and fixed with 150 μl 2% formalin for FACS staining to verify CFSE incorporation into the cells. The remaining cells were resuspended in culture media and recultured. Cells samples were taken for FACS analysis after the 24 hr and 48 hr time points.

Animals. Female BALB/C mice at 4-6 weeks of age were purchased from Jackson Laboratories. The 4T07 cells express the H-2 (the murine equivalent of human MHC) haplotype d, as do the BALB/C mice. Mice were maintained in the Emory University animal facility according to regulations of the Institutional Animal Use Committee.

Direct Challenge of Mice with Transfected Tumor Cells and Memory Study. Mice (n=5/group) were challenged subcutaneously (s.c.) with wild-type 4T07 or transfected 4T07-B7, IL-2, IL-12, B7/IL-2 or B7/IL-12 cells (all 2×10⁵ cells in 10 μl PBS). Cells were harvested by dissociation with 0.05% trypsin/EDTA, were washed 1× with PBS, and were resuspended at 2×10⁶ cells/ml. The mice's backs were shaved, and the mice were anesthetized with isofurane (Emory University Hospital Pharmacy). Mice were injected s.c. in the rear flank and were monitored daily. Tumor size was measured using Vernier calipers every 2^{nd}-3^{rd} day by 2×2 perpendicular measurements, and tumor size (mm²) was calculated by multiplying the two diameters. Mice were euthanized when the tumor size reached >2 cm. 30-33 days after the initial challenge, tumor-free mice in the experimental groups were rechallenged on the opposite hind flank with wild-type 4T07 cells (2×10⁵ in 100 μl PBS). Mice were monitored for tumor growth.

Isolation of Mice Spleens. On Day 37 of direct challenge, the mice from the wild-type 4TO7 control group were sacrificed (CO₂ euthanasia), and their spleens were dissected. Spleen mass was measured and photographs taken. Spleens were broken up in PBS using forceps and dissecting scissors. The T cell suspension in PBS was separated from the solid fragments of spleen and was centrifuged at 1200 rpm for 7 minutes at 4° C. Cell pellet of T cells was resuspended in freezing media and T cells were frozen for future analysis. Spleens were also isolated from unchallenged, normal BALB/C mice (n=3) for comparison to the spleens from the challenged mice. T cells were also isolated and saved for future proliferation assays.

### Results

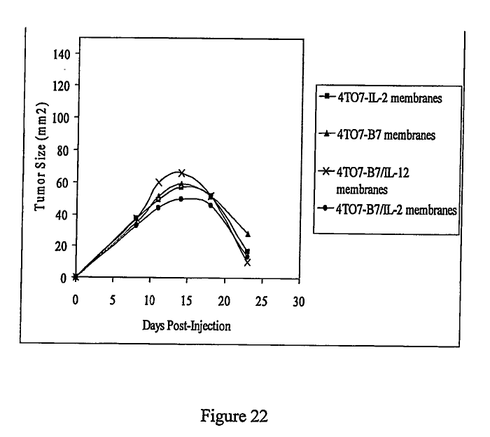

Using established, selected and verified transfected tumor cell lines, this example shows that the method of vaccination described herein can be used to prevent and/or treat breast cancer. Some of the results were summarized in FIG. 22, which shows (1) that primary challenge of transfected tumor cells in mice led to tumor rejection, (2) that secondary challenge of tumor-free mice with wild-type cells showed immunity was induced, and (3) that a combination of cytokine such as IL-12 and a costimulatory molecule such as B7 can be most effective

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A tumor therapeutic composition or tumor vaccine effective for preventing a tumor from growth, causing the tumor to regress, or providing antitumor immunity, comprising glycosyl-phosphatidylinositol (GPI)-anchored immunostimulatory or costimulatory molecules and a material selected from the group consisting of tumor cells, tumor cell membranes, biocompatible microparticles encapsulating a tumor antigen or peptide, tumor lysate or tumor membranes, inactivated or partially attenuated virus, bacteria and virus-like particles, and combinations thereof,
wherein the costimulatory molecule is not B7-1 or B7-2, and
wherein the immunostimulatory molecule is not interleukin-6 (IL-6). interleukin-12 (IL-12), or granulocyte-macrophage colony-stimulating factor (GM-CSF).

2. The tumor therapeutic composition or tumor vaccine of claim 1, wherein the GPI-anchored immunostimulatory or costimulatory molecules are molecules of a cytokine or a combination of cytokines.

3. The tumor therapeutic composition or tumor vaccine of claim 1, wherein the GPI-anchored immunostimulatory or costimulatory molecules are selected from the group consisting of IL-2, IL-4, ICAM-1, CD40L, IL-15, IL-18, IL-19, and combinations thereof.

4. The tumor therapeutic composition or tumor vaccine of claim 1, wherein the tumor antigen or peptide is selected from the group consisting of mutated p53, antigenic peptides derived from p53, melanoma specific tumor antigens such as MAGE family proteins (eg MAGE-1) and peptides (eg AARAVFLAL) derived from these proteins, and combinations thereof.

5. The tumor therapeutic composition or tumor vaccine of claim 1, comprising tumor cells or tumor cell membranes.

6. The tumor therapeutic composition or tumor vaccine of claim 1 wherein the biocompatible microparticles are biodegradable.

7. The tumor therapeutic composition or tumor vaccine of claim 4 wherein the biocompatible microparticles comprise a polymer selected from the group consisting of polyvinyl alcohols, polyvinyl ethers, polyamides, polyvinyl esters, polyvinylpyrrolidone, polyglycolides, polyurethanes, alkyl celluloses, cellulose esters, hydroxypropyl derivatives of celluloses and cellulose esters, preformed polymers of poly alkyl acrylates, polyethylene, polystyrene, polyactic acid, polyglycolic acid, poly(lactide-co-glycolide), polycaprolactones, polybutyric acids, polyvaleric acid and copolymers thereof, alginates, chitosans, gelatin, albumin, zein and combinations thereof.

8. The tumor therapeutic composition or tumor vaccine of claim 1.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

9. The tumor therapeutic composition or tumor vaccine of claim 2.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

10. The tumor therapeutic composition or tumor vaccine of claim 3.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

11. The tumor therapeutic composition or tumor vaccine of claim 4.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

12. The tumor therapeutic composition or tumor vaccine of claim 5.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

13. The tumor therapeutic composition or tumor vaccine of claim 6.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

14. The tumor therapeutic composition or tumor vaccine of claim 7.
wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

15. A vaccine or therapeutic composition comprising glycosyl-phosphatidylinositol (GPI)-anchored cytokine selected from GPI-IL-2, GPI-IL-12, and a combination thereof with a costimulatory molecule.

16. The vaccine or therapeutic composition of claim 15 wherein
wherein the costimulatory molecule is selected from the group consisting of CD40L, ICAM-1, ICAM-2, ICAM-3 and a combination thereof.

17. A therapeutic composition or vaccine comprising glycosyl-phosphatidylinositol (GPI)-anchored immunostimulatory or costimulatory molecules and a material selected from the group consisting of biocompatible microparticles, inactivated or partially attenuated virus, bacteria, and virus-like particles, and combinations thereof.

18. The therapeutic composition or vaccine of claim 17, wherein the GPI-anchored immunostimulatory or costimulatory molecules are molecules of a cytokine or a combination of cytokines.

19. The therapeutic composition or vaccine of claim 17, wherein the GPI-anchored immunostimulatory or costimulatory molecules are selected from the group consisting of IL-2, IL-4, IL-6, IL-12, ICAM-1, ICAM-2, ICAM-3, B7-1, B7-2, CD40L, IL-15, IL-18, IL-19, granulocyte-macrophage colony stimulating factor (GM-CSF), and combinations thereof.

20. The therapeutic composition or vaccine of claim 17, wherein the biocompatible microparticles are biodegradable.

21. The therapeutic composition or vaccine of claim 17 wherein the biocompatible microparticles comprise a polymer selected from the group consisting of polyvinyl alcohols, polyvinyl ethers, polyamides, polyvinyl esters, polyvinylpyrrolidone, polyglycolides, polyurethanes, alkyl celluloses, cellulose esters, hydroxypropyl derivatives of celluloses and cellulose esters, preformed polymers of poly alkyl acrylates, polyethylene, polystyrene, polyactic acid, polyglycolic acid, poly(lactide-co-glycolide), polycaprolactones, polybutyric acids, polyvaleric acid and copolymers thereof, alginates, chitosans, gelatin, albumin, zein and combinations thereof.

22. The therapeutic composition or vaccine of claim 17 which is effective for treating a disease or disorder selected from the group consisting of viral diseases, bacterial diseases, parasitic diseases, autoimmune disorders, transplant rejection, and combinations thereof.

23. A method of treating a tumor or providing antitumor immunity, comprising:
applying to a patient a tumor therapeutic composition or tumor vaccine as defined in a claim 1,
wherein the tumor therapeutic composition or tumor vaccine prevents or delays growth of the tumor, causes the tumor to regress or provides antitumor immunity against the tumor.

24. The method of claim 23 wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

25. A method of the treatment or vaccination for a disease or disorder, comprising:
applying to a patient a therapeutic composition or vaccine as defined in claim 17,
wherein the therapeutic composition or prevents or ameliorates the disease or disorder.

26. The method of claim 25 wherein the disease or disorder is selected from the group consisting of viral diseases, bacterial diseases, parasitic diseases, autoimmune disorders, transplant rejection, and combinations thereof.

27. A method of making a therapeutic composition or vaccine, comprising:
providing glycosyl-phosphatidylinositol (GPI)-anchored immunostimulatory or costimulatory molecules,
providing a material selected from the group consisting of tumor cells, tumor cell membranes, biocompatible microparticles, biocompatible microparticles encapsulating a tumor antigen or peptide, tumor lysate or tumor membranes, inactivated or partially attenuated virus, bacteria and virus-like particles, and combinations thereof, and
transferring the GPI-anchored immunostimulatory or costimulatory molecules onto the surface of the material by protein transfer,
wherein the costimulatory molecule is not B7-1 or B7-2 and
wherein the immunostimulatory molecule is not interleukin-6 (IL-6), interleukin-12 (IL-12), or granulocyte-macrophage colony-stimulating factor (GM-CSF).

28. The method of claim 27, wherein the GPI-anchored immunostimulatory or costimulatory molecules are molecules of a cytokine or a combination of cytokines.

29. The method of claim 27, wherein the GPI-anchored immunostimulatory or costimulatory molecules are selected from the group consisting of IL-2, IL-4, ICAM-1, ICAM-2, ICAM-3, CD40L, IL-15, IL-18, and combinations thereof.

30. The method of claim 27, wherein the material is a plurality of biocompatible microparticles encapsulating a tumor antigen or peptide selected from the group consisting of mutated p53, antigenic peptides derived from p53, melanoma specific tumor antigens such as MAGE family proteins (eg MAGE-1) and peptides (eg AARAVFLAL) derived from these proteins, and combinations thereof.

31. The method of claim 27, wherein the material is a plurality of tumor cells or tumor cell membranes.

32. The method of claim 27 wherein the material is a plurality of biocompatible particles which are biodegradable.

33. The method of claim 30, wherein the biocompatible microparticles comprise a polymer selected from the group consisting of polyvinyl alcohols, polyvinyl ethers, polyamides, polyvinyl esters, polyvinylpyrrolidone, polyglycolides, polyurethanes, alkyl celluloses, cellulose esters, hydroxypropyl derivatives of celluloses and cellulose esters, preformed polymers of poly alkyl acrylates, polyethylene, polystyrene, polyactic acid, polyglycolic acid, poly(lactide-co-glycolide), polycaprolactones, polybutyric acids, polyvaleric acid and copolymers thereof, alginates, chitosans, gelatin, albumin, zein and combinations thereof.

34. The method of claim 27 wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

35. A method of treating a tumor or providing antitumor immunity, comprising:
applying to a patient a tumor therapeutic composition or tumor vaccine as defined in claim 15,
wherein the tumor therapeutic composition or tumor vaccine prevents or delays growth of the tumor, causes the tumor to regress or provides antitumor immunity against the tumor.

36. The method of claim 35 wherein the tumor is selected from the group consisting of breast cancer, prostate cancer, lung cancer, melanoma, liver cancer, leukemia, lymphoma, myeloma, colorectal cancer, gastric cancer, bladder carcinoma, esophageal carcinoma, head & neck squamous-cell carcinoma, sarcomas, kidney cancers, ovarian and uterus cancers, adenocarcinoma, gilioma, and plasmacytoma, and combinations thereof.

37. A tumor therapeutic composition or tumor vaccine effective for preventing a tumor from growth, causing the tumor to regress, or providing antitumor immunity, comprising glycosyl-phosphatidylinositol (GPI)-anchored immunostimulatory or costimulatory molecules and a plurality of biocompatible microparticles encapsulating a tumor antigen, peptide, or a combination thereof.

38. The tumor therapeutic composition or tumor vaccine of claim 37, wherein the GPI-anchored immunostimulatory or costimulatory molecules are molecules of a cytokine or a combination of cytokines.

39. A method of treating a tumor or providing antitumor immunity, comprising:
applying to a patient a tumor therapeutic composition or tumor vaccine as defined in claim 37,
wherein the tumor therapeutic composition or tumor vaccine prevents or delays growth of the tumor, causes the tumor to regress or provides antitumor immunity against the tumor.
